(19) United States
(12) Patent Application Publication
(10) Pub. No.: US 2009/0018128 A1
(43) Pub. Date: Jan. 15, 2009

(54) Compounds

(76) Inventors: Andrew John Eatherton, Essex (GB); Gerard Martin Paul Giblin, Essex (GB); William Leonard Mitchell, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/162,824

(22) PCT Filed: Jan. 30, 2007
(86) PCT No.: PCT/EP07/50916

§ 371 Date: Jul. 31, 2008

(30) Foreign Application Priority Data
Feb. 1, 2006 (GB) 0602042.4

(51) Int. Cl. C07D 487/04; A61K 31/437; A61K 31/5377; A61P 29/00; A61P 19/02; A61P 19/10

(52) U.S. Cl. 514/233.2; 544/119; 544/349; 514/249

(57) ABSTRACT

The present invention relates to novel pyrrolopyrazine derivatives, pharmaceutical compositions containing these compounds and their use in the treatment of diseases, particularly pain, which diseases are caused directly or indirectly by an increase or decrease in activity of the cannabinoid receptor.

### COMPOUNDS

The present invention relates to novel pyrrolopyrazine derivatives, pharmaceutical compositions containing these compounds and their use in the treatment of diseases, particularly pain, which diseases are caused directly or indirectly by an increase or decrease in activity of the cannabinoid receptor.

Cannabinoids are a specific class of psychoactive compounds present in Indian cannabis (Cannabis sativa), including about sixty different molecules, the most representative being cannabinol, cannabidiol and several isomers of tetrahydrocannabinol. Knowledge of the therapeutic activity of cannabis dates back to the ancient dynasties of China, where, 5,000 years ago, cannabis was used for the treatment of asthma, migraine and some gynaecological disorders. These uses later became so established that, around 1850, cannabis extracts were included in the US Pharmacopaeia and remained there until 1947.

Cannabinoids are known to cause different effects on various systems and/or organs, the most important being on the central nervous system and on the cardiovascular system. These effects include alterations in memory and cognition, euphoria, and sedation. Cannabinoids also increase heart rate and vary systemic arterial pressure. Peripheral effects related to bronchial constriction, immunomodulation, and inflammation have also been observed. The capability of cannabinoids to reduce intraocular pressure and to affect respiratory and endocrine systems is also well documented. See e.g. L. E. Hollister, Health Aspects of Cannabis, Pharmacological Reviews, Vol. 38, pp. 1-20, (1986). More recently, it was found that cannabinoids suppress the cellular and humoral immune responses and exhibit antiinflammatory properties. Wirth et al., Antiinflammatory Properties of Cannabichrome, Life Science, Vol. 26, pp. 1991-1995, (1980).

In spite of the foregoing benefits, the therapeutic use of cannabis is controversial, both due to its relevant psychoactive effects (causing dependence and addiction), and due to manifold side effects that have not yet been completely clarified. Although work in this field has been ongoing since the 1940's, evidence indicating that the peripheral effects of cannabinoids are directly mediated, and not secondary to a CNS effect, has been limited by the lack of receptor characterization, the lack of information concerning an endogenous cannabinoid ligand and, until recently, the lack of receptor subtype selective compounds.

The first cannabinoid receptor was found to be mainly located in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,” Nature, Vol. 346, pp. 561-564 (1990). The second cannabinoid receptor (“CB2”) was identified in the spleen, and was assumed to modulate the non psychoactive effects of the cannabinoids. See Munro et al., “Molecular Characterization of a Peripheral Receptor for Cannabinoids,” Nature, Vol. 365, pp. 61-65 (1993).

The foregoing indications and the preferential localization of the CB2 receptor in the immune system confirms a specific role of CB2 in modulating the immune and antiinflammatory response to stimuli of different sources.

The total size of the patient population suffering from pain is vast (almost 300 million), dominated by those suffering from back pain, osteo-arthritic pain and post-operative pain. Neuropathic pain (associated with neuronal lesions such as those induced by diabetes, HIV, herpes infection, or stroke) occurs with lower, but still substantial prevalence, as does cancer pain.

The pathogenic mechanisms that give rise to pain symptoms can be grouped into two main categories:
those that are components of inflammatory tissue responses (Inflammatory Pain);
those that result from a neuronal lesion of some form (Neuropathic Pain).

Chronic inflammatory pain consists predominantly of osteoarthritis, chronic low back pain and rheumatoid arthritis. The pain results from acute and on-going injury and/or inflammation. There may be both spontaneous and provoked pain.

There is an underlying pathological hypersensitivity as a result of physiological hyperexcitability and the release of inflammatory mediators which further potentiate this hyperexcitability. CB2 receptors are expressed on inflammatory cells (T cells, B cells, macrophages, mast cells) and mediate immune suppression through inhibition of cellular interaction/inflammatory mediator release. CB2 receptors may also be expressed on sensory nerve terminals and therefore directly inhibit hyperalgesia.

More recently, data suggests a role for CB2 receptor activation in the CNS. Until recently the CB2 receptor was thought to be restricted to the periphery, however emerging data suggests inflammatory pain-mediated induction of CB2 receptor expression in rat spinal cord which coincides with the appearance of activated microglia (Zhang et. al., 2003). Furthermore CB2 receptor agonists have been shown to reduce mechanically evoked responses and wind-up of wide dynamic range neurones in spinal cord dorsal horn in animal models of inflammatory pain (Zhang et. al., 2003, Eur J. Neurosci. 17: 2750-2754, Nackley et. al., 2004, J. Neurophys. 92: 3562-3574, Elmes et. al., 2004, Eur. J. Neurosci. 20: 2311-2320).

The role of CB2 in immunomodulation, inflammation, osteoporosis, cardiovascular, renal and other disease conditions is now being examined.

Based on the foregoing, there is a need for compounds which have activity against the CB2 receptor. Thus, CB2 modulators are believed to offer an unique approach toward the pharmacotherapy of immune disorders, inflammation, osteoporosis, renal ischemia and other pathophysiological conditions.

The present invention provides novel pyrollopyrazine derivatives of formula (I) and pharmaceutically acceptable derivatives thereof, pharmaceutical compositions containing these compounds or derivatives, and their use as CB2 receptor modulators, which are useful in the treatment of a variety of disorders.

The present invention further comprises a method for treating disease mediated by CB2 receptors in an animal, including humans, which comprises administering to an animal in need thereof an effective, non-toxic amount of a compound of formula (I) or a pharmaceutically acceptable derivative thereof.

In light of the fact that cannabinoids act on receptors capable of modulating different functional effects, and in view of the low homology between CB2 and CB1, a class of drugs selective for the specific receptor sub-type is desirable. The natural or synthetic cannabinoids currently available do not fulfil this function because they are active on both receptors.

In one embodiment the present invention includes compounds which are capable of selectively modulating the receptors for cannabinoids and therefore the pathologies associated with such receptors.

The invention provides compounds of formula (I):

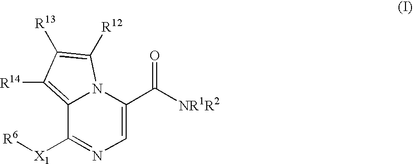

wherein:

X₁ is NR⁴, O, S, SO or SO₂;

R¹ is selected from hydrogen, C₁₋₆ alkyl, C₃₋₆cycloalkyl and halosubstitutedC₁₋₆ alkyl;

R² is hydrogen or (CH₂)ₘR³ where m is 0 or 1;

or R¹ and R² together with N to which they are attached form an optionally substituted 4- to 8-membered non-aromatic heterocyclyl ring;

R³ is a 4- to 8-membered non-aromatic heterocyclyl group, a C₃₋₈ cycloalkyl group, a straight or branched C₁₋₁₀ alkyl, a C₂₋₁₀alkenyl, a C₃₋₈cycloalkenyl, a C₂₋₁₀alkynyl, a C₃₋₈cycloalkynyl or phenyl group, any of which can be unsubstituted or substituted, or R⁵;

R⁴ is selected from hydrogen, C₁₋₆ alkyl, C₃₋₆ cycloalkyl and halosubstitutedC₁₋₆ alkyl;

R⁵ is

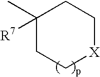

wherein p is 0, 1 or 2, and X is CH₂, O, S, or SO₂;

R⁶ is unsubstituted or substituted phenyl, unsubstituted or substituted C₃₋₆cycloalkyl or an unsubstituted or substituted 4- to 8-membered non-aromatic heterocyclyl ring;

R⁷ is OH;

R¹² is hydrogen or C₁₋₆alkyl;

R¹³ is hydrogen or C₁₋₆alkyl;

R¹⁴ is hydrogen or C₁₋₆alkyl;

and pharmaceutically acceptable derivatives thereof.

In one embodiment X₁ is NR⁴. In another embodiment X₁ is O.

In one embodiment R¹ is hydrogen.

In one embodiment R² is (CH₂)ₘR³ where m is 0 or 1.

In another embodiment R¹ and R² taken together with the N to which they are attached form an optionally substituted 4- to 8-membered non-aromatic heterocyclyl ring. In this embodiment the ring may optionally contain 1, 2, 3 or 4 further hetero atoms. The ring may be saturated or unsaturated. In a further embodiment the further hetero atoms are selected from oxygen or sulphur. An example of a 4-membered heterocyclyl ring is azetidinyl. Examples of a 5-membered heterocyclyl ring are pyrrolidinyl and pyrazolidinyl. Examples of 6-membered heterocyclyl rings are morpholinyl, piperidinyl, tetrahydropyridinyl, thiomorpholine-s,s-dioxide, thiomorpholinyl and thiomorpholinyl-s-oxide. Examples of a 7-membered heterocyclyl ring are azapine or oxapine. Examples of 8-membered heterocyclyl rings are azacyclooctanyl, azaoxacyclooctanyl or azathiacyclooctanyl. In yet another embodiment R¹ and R² together with the nitrogen to which they are attached form a morpholinyl, pyrrolidinyl or piperidinyl ring.

In one embodiment R³ is a 4- to 8-membered non-aromatic heterocyclyl group. In another embodiment R³ is an unsubstituted or substituted C₁₋₆ alkyl group.

In one embodiment R⁴ is C₁₋₆ alkyl or hydrogen, for example methyl or hydrogen. In another embodiment R⁴ is hydrogen.

In one embodiment R⁶ is an unsubstituted or substituted phenyl. In another embodiment R⁶ is an unsubstituted or substituted 4- to 8-membered non-aromatic heterocyclyl group.

When R³ or R⁶ are independently selected from a non-aromatic heterocyclyl group, the ring may contain 1, 2, 3, or 4 hetero atoms. In one embodiment the hetero atoms are selected from oxygen, nitrogen or sulphur. Examples of 4-membered groups are 2- or 3-azetidinyl, oxetanyl, thioxetanyl, thioxetanyl-s-oxide and thioxetanyl-s,s-dioxide. Examples of 5-membered heterocyclyl groups in this instance include dioxolanyl, pyrrolidinyl, tetrahydrofuranyl, tetrahydrothiophenyl, tetrahydrothiophenyl-s,s-dioxide and tetrahydrothiophenyl-s-oxide. Examples of 6-membered heterocyclyl groups are morpholinyl, piperidinyl, piperazinyl, tetrahydropyranyl, tetrahydrothiopyranyl, tetrahydrothiopyranyl-s,s-dioxide, thiomorpholinyl, thiomorpholinyl-s,s-dioxide, tetrahydropyridinyl, dioxanyl, tetrahydrothiopyran-1,1-dioxide and tetrahydrothiopyran-1-oxide. Examples of a 7-membered heterocyclyl ring are azapine or oxapine. Examples of 8-membered groups are azacyclooctanyl, azaoxacyclooctanyl or azathiacyclooctanyl, oxacylcooctanyl, thiacyclooctanyl and azathiacyclooctanyl-s-oxide, azathiacyclooctanyl-s,s-dioxide, thiacyclooctanyl-s,s-dioxide, and thiacyclooctanyl-s-oxide.

In one embodiment R¹² is methyl or ethyl.

In one embodiment R¹³ is methyl or hydrogen.

In one embodiment R¹⁴ is methyl or hydrogen.

When R¹ and R² together with N to which they are attached form a 4- to 8-membered non-aromatic heterocyclyl ring which is substituted, or when R³ is substituted, there can be 1, 2, or 3 substituents. The substituent or substituents may be selected from: C₁₋₆ alkyl, C₁₋₆ alkoxy, a hydroxy group, halosubstituted C₁₋₆alkyl e.g. trifluoromethyl, halosubstituted C₁₋₆alkoxy e.g. trifluoromethyoxy, a cyano group, halo or a sulfonyl group, methylsulfonyl, NR^{8a}R^{8b}, CONH₂, NHCOCH₃, (═O), COOH, CONHCH₃, CON(CH₃)₂ and NHSO₂CH₃ wherein R^{8a} and R^{8b} are independently selected from hydrogen or C₁₋₆alkyl.

When R⁶ is substituted, it may be substituted by 1, 2 or 3 substituents, the substituent or substituents may be selected from: C₁₋₆ alkyl, halosubstitutedC₁₋₆ alkyl e.g. trifluoromethyl, C₁₋₆ alkoxy, a hydroxy group, a cyano group, halo and halosubstituted C₁₋₆ alkoxy e.g. trifluoromethyloxy. In one embodiment R⁶ is substituted by 1 or 2 substituents. In another embodiment R⁶ is substituted by halo, cyano, methyl, trifluoromethyl, methoxy or trifluoromethoxy. In a further embodiment R⁶ is substituted by halo.

When R⁶ is phenyl it may be substituted by two groups which together form a fused ring. In one embodiment the fused ring is a 5-membered non-aromatic heterocyclyl ring such as tetrahydrofuranyl.

In one embodiment the invention is compounds of formula (Ia);

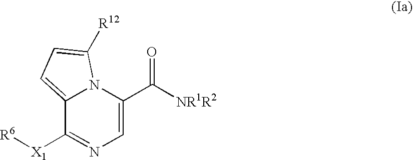

wherein

X₁ is NR⁴;

R¹ is hydrogen;

R² is (CH₂)ₘR³ where m is 0 or 1;

or R¹ and R² together with N to which they are attached form a morpholinyl, pyrrolidinyl, or piperidinyl ring of which may be unsubstituted or substituted;

R³ is an unsubstituted or substituted straight or branched C₁₋₆ alkyl;

R⁴ is hydrogen or methyl,

R⁶ is unsubstituted or substituted phenyl;

R¹² is methyl;

and pharmaceutically acceptable derivatives thereof.

In certain embodiments compounds of formula (I) show selectivity for CB2 over CB1.

In one embodiment compounds of formula (I) have an EMR value at the cloned human cannabinoid CB2 receptor of at least 5 times the EMR value at the cloned human cannabinoid CB1 receptor. In another embodiment compounds of formula (I) have an EMR value at the cloned human cannabinoid CB2 receptor of at least 10 times the EMR value at the cloned human cannabinoid CB1 receptor. EMR is the equieffective molar ratio and values may be calculated from the equation set out hereinbelow.

Compounds of formula (I) may be more potent and/or more soluble and/or more bioavailable and/or produce a more linear increase in exposure when the compounds are orally administered to a mammal than earlier published compounds which are agonists of CB2.

The invention is described using the following definitions unless otherwise indicated.

The term “pharmaceutically acceptable derivative” means any pharmaceutically acceptable salt, ester, salt of such ester or solvate (including solvates of salts, esters, or salts of esters) of the compounds of formula (I), or any other compound which upon administration to the recipient is capable of providing (directly or indirectly) a compound of formula (I) or an active metabolite or residue thereof. In one embodiment the pharmaceutically acceptable derivative is a salt or solvate of compound of formula (I).

It will be appreciated by those skilled in the art that compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts, esters, salts of such esters or solvates (including solvates of salts, esters, or salts of esters) referred to above will be physiologically acceptable salts, esters, salts of such esters or solvates (including solvates of salts, esters, or salts of esters) but other salts, esters, salts of such esters or solvates (including solvates of salts, esters, or salts of esters) may find use, for example in the preparation of compounds of formula (I) and the physiological acceptable salts, esters, salts of such esters or solvates (including solvates of salts, esters, or salts of esters) thereof. Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The terms ‘halogen or halo’ are used to represent fluorine, chlorine, bromine or iodine.

The term ‘alkyl’ as a group or part of a group means a straight or branched chain alkyl group or combinations thereof, for example a methyl, ethyl, n-propyl, i-propyl, n-butyl, s-butyl, t-butyl, i-butyl, pentyl, hexyl, 1,1-dimethylethyl, heptyl, octyl, nonyl, decyl or combinations thereof.

The term ‘alkoxy’ as a group or as part of a group means a straight, branched or cyclic chain alkyl group having an oxygen atom attached to the chain, for example a methoxy, ethoxy, n-propoxy, i-propoxy, n-butoxy, s-butoxy, t-butoxy group, i-butoxy, pentoxy, hexyloxy group, cyclopentoxy or cyclohexyloxy group.

The term ‘cycloalkyl’ means a closed saturated ring, for example cyclopropyl, cyclobutyl, cyclopentyl, cyclohexyl or cycloheptyl, or cyclooctyl.

The term ‘alkenyl’ means as a group or part of a group means a straight or branched chain carbon chain or combinations thereof containing 1 or more double bonds, for example butenyl, pentenyl, hexenyl or heptenyl, or octenyl.

The term ‘cycloalkenyl’ means a closed non-aromatic carbon ring containing 1 or more double bonds, for example cyclobutenyl, cyclopentenyl, cyclohexenyl or cycloheptenyl, or cyclooctenyl.

The term ‘alkynyl’ as a group or part of a group means a straight or branched chain carbon chain or combinations containing 1 or more triple carbon bonds for example ethynyl, propynyl, butynyl, pentynyl, hexynyl or combinations thereof.

The term ‘cycloalkynyl’ means a closed non-aromatic carbon ring containing 1 or more triple carbon bonds for example cyclopropynyl, cyclobutynyl, cyclopentynyl, cyclohexynyl or combinations thereof.

The term ‘aryl’ means a 5- or 6-membered aromatic ring, for example phenyl, or a 7- to 12-membered bicyclic ring system where at least one of the rings is aromatic, for example naphthyl.

The present invention also provides processes for the preparation of compounds of formula (I) or pharmaceutically acceptable salts or solvates thereof.

Compounds of formula (I) wherein X₁ is NR⁴ may be prepared as set out in Scheme 1:

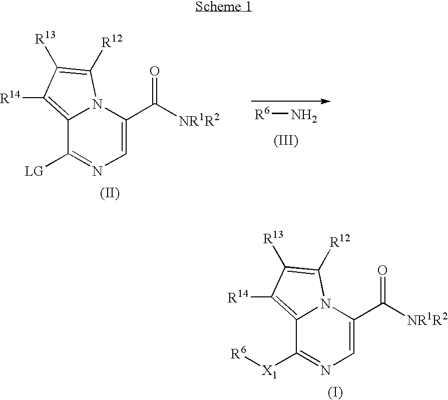

wherein R¹, R², R⁶, R¹², R¹³ and R¹⁴ are as defined for compounds of formula (I) above or are groups convertible thereto and LG is a suitable leaving group, such as a halogen atom (e.g. fluorine, chlorine, bromine or iodine) or triflate group. This process typically comprises the use of a suitable acid such as methanesulfonic acid, in a suitable solvent such as 1,4-dioxane at elevated temperature such as 180° C. and under microwave conditions. When the group of formula (III) is an amine group i.e. R⁶ is a unsubstituted or substituted C₃₋₆cycloalkyl or an unsubstituted or substituted 4- to 8-membered non-aromatic heterocyclyl ring, the use of an acid and a separate solvent is optional.

Compounds of formula (I) wherein X₁ is O may be prepared as set out in Scheme 2:

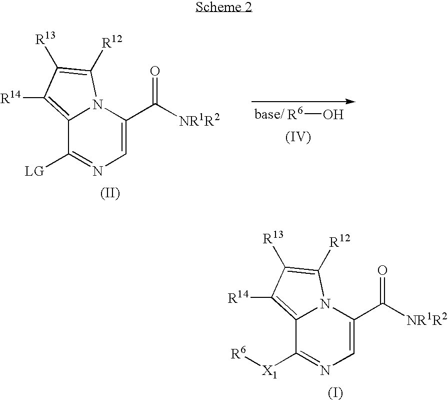

wherein R¹, R², R⁶, R¹², R¹³ and R¹⁴ are as defined for compounds of formula (I) above or are groups convertible thereto and LG is a suitable leaving group, such as a halogen atom (e.g. fluorine, chlorine, bromine or iodine) or triflate group. Suitably the group of formula (IV) is first deprotonated by a strong base such as sodium hydride. This process typically comprises the use of a suitable solvent such as N,N-dimethylformamide and is carried out at elevated temperature such as 180° C. under microwave conditions.

Compounds of formula (I) wherein X₁ is S, SO or SO₂ may be prepared as set out in Scheme 3:

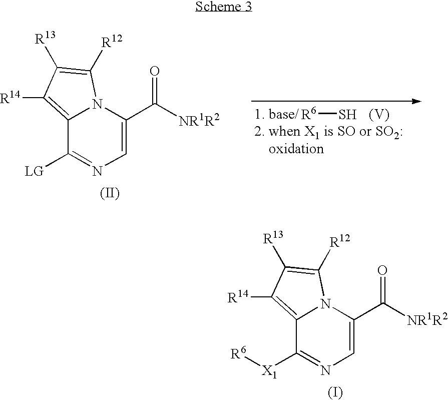

wherein R¹, R², R⁶, R¹², R¹³ and R¹⁴ are as defined for compounds of formula (I) above or are groups convertible thereto and LG is a suitable leaving group, such as a halogen atom (e.g. fluorine, chlorine, bromine or iodine) or triflate group. Suitably the group of formula (V) is first deprotonated by a strong base such as sodium hydride. This process typically comprises the use of a suitable solvent such as N,N-dimethylformamide and is carried out at elevated temperature such as 180° C. under microwave conditions. Suitable oxidising agents for use when X₁ is SO or SO₂ include 3-chloroperoxybenzoic acid and a suitable solvent for use in the oxidation step is dichloromethane.

Compounds of formula (II) may be prepared as set out in Scheme 4:

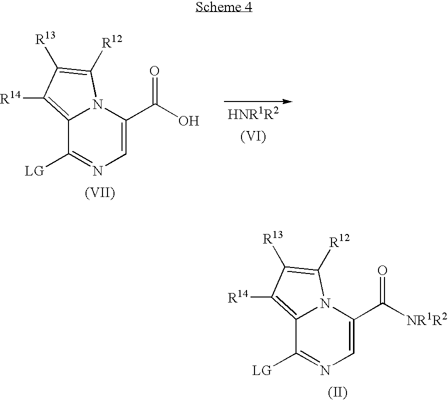

wherein R¹, R², R¹², R¹³ and R¹⁴ are as defined above or groups convertible thereto and LG is a suitable leaving group, such as a halogen atom (e.g. fluorine, chlorine, bromine or iodine) or triflate group. This process typically comprises the use of a suitable tertiary base e.g. N-ethyl diisopropylamine or N-ethylmorpholine and a coupling agent e.g. O-(1H-benzotriazol-1-yl)-N,N,N′,N′-tetramethyluronium hexafluorophosphate or a combination of 1-hydroxybenzotriazole hydrate and N-(3-dimethylaminopropyl)-N′-ethylcarbodiimide. A suitable solvent for carrying out this coupling reaction is N,N-dimethylformamide.

Compounds of formula (VI) may be prepared according to Scheme 5:

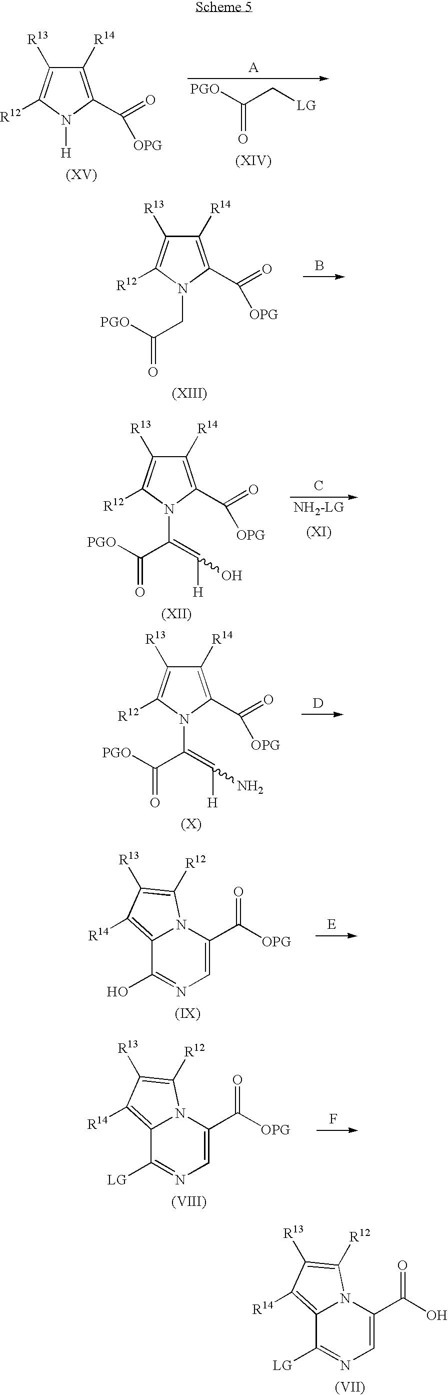

wherein R¹, R², R⁶, R¹⁰, R¹², R¹³ and R¹⁴ are as defined for compounds of formula (I) above or are groups convertible thereto, LG is a suitable leaving group, such as a halogen atom (e.g. fluorine, chlorine, bromine or iodine) or triflate group, and PG is a is a protecting group such as C₁₋₆alkyl for example methyl or ethyl.
Step A: react (XV) and (XIV) (e.g. ethylbromoacetate) in the presence of a base e.g. sodium hydride to give (XIII).
Step B: react (XIII) and e.g. ethylformate in the presence of a strong base to give (XII)
Step C: react (XII) and (XI) e.g. ammonium acetate under reflux to give (X).
Step D: ring formation in the presence of a strong base e.g. sodium tert-butoxide at elevated temperature (e.g. 160° C.) under microwave to give (IX).
Step E: add leaving group e.g. by reaction of (IX) with phenyldichlorophosphate at elevated temperature (e.g. 170° C.).
Step F: deprotect e.g. using 2N sodium hydroxide.

For preparation of intermediate (XV) when R¹² is methyl and R¹³ and R¹⁴ are hydrogen was see: Curran, Timothy P.; Keaney, Meghan T. Journal of Organic Chemistry (1996), 61 (25), 9068-9069. For preparation of intermediate (XV) when R¹² and R¹³ are hydrogen and R¹⁴ is methyl or ethyl or when R¹² and R¹⁴ are hydrogen and R¹³ is methyl or ethyl or when R¹² is ethyl and R¹³ and R¹⁴ are hydrogen see: Lash, Timothy D.; Hoehner, Michael C. Journal of Heterocyclic Chemistry (1991), 28 (7), 1671-1676.

Compounds of formula (III)-(VI), (XI) and (XIV) are either known in the literature or can be prepared by analogous methods.

Compounds of formula (II), (VII)-(X), (XII) and (XIII) are novel intermediates and form a further aspect of the present invention.

It is to be understood that the present invention encompasses all isomers of compounds of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The subject invention also includes isotopically-labeled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I) and following of this invention can generally be prepared by carrying out the procedures disclosed in the Schemes and/or in the Examples below, by substituting a readily available isotopically labeled reagent for a non-isotopically labeled reagent.

Compounds of formula (I) may be prepared in crystalline or non-crystalline form, and, if crystalline, may optionally be solvated. References to solvates herein include hydrates. This invention includes within its scope stoichiometric solvates (including hydrates) as well as compounds containing variable amounts of water and/or solvent.

In view of their ability to bind to the CB2 receptor, it is believed that compounds of the invention will be useful in the treatment of the disorders that follow. Thus, compounds of formula (I) and their pharmaceutically acceptable derivatives may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may also have disease modification or joint structure preservation properties in multiple sclerosis, rheumatoid arthritis, osteo-arthritis, rheumatoid spondylitis, gouty arthritis and juvenile arthritis.

Compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of formula (I) and their pharmaceutically acceptable derivatives may also be useful in the treatment of fever.

Compounds of formula (I) and their pharmaceutically acceptable derivatives may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

Compounds of formula (I) and their pharmaceutically acceptable derivatives may also be useful in the treatment of bladder hyperrelexia following bladder inflammation.

Compounds of formula (I) and their pharmaceutically acceptable derivatives may also be useful in the treatment of immunological diseases such as autoimmune diseases, immunological deficiency diseases or organ transplantation. The compounds of formula (I) and their pharmaceutically acceptable derivatives may also be effective in increasing the latency of HIV infection.

Compounds of formula (I) and their pharmaceutically acceptable derivatives may also be useful in the treatment of diseases of abnormal platelet function (e.g. occlusive vascular diseases).

Compounds of formula (I) and their pharmaceutically acceptable derivatives may also be useful in the treatment of neuritis, heart burn, dysphagia, pelvic hypersensitivity, urinary incontinence, cystitis or pruritis.

Compounds of formula (I) and their pharmaceutically acceptable derivatives may also have diuretic action.

Compounds of formula (I) and their pharmaceutically acceptable derivatives may also be useful in the treatment of impotence or erectile dysfunction.

Compounds of formula (I) and their pharmaceutically acceptable derivatives may also be useful for attenuating the hemodynamic side effects of non-steroidal anti-inflammatory drugs (NSAID's) and cyclooxygenase-2 (COX-2) inhibitors.

Compounds of formula (I) and their pharmaceutically acceptable derivatives may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

Compounds of formula (I) and their pharmaceutically acceptable derivatives may also be useful in neuroprotection and in the treatment of neurodegeneration following stroke, cardiac arrest, pulmonary bypass, traumatic brain injury, spinal cord injury or the like.

Compounds of formula (I) and their pharmaceutically acceptable derivatives may also be useful in the treatment of tinnitus.

Compounds of formula (I) and their pharmaceutically acceptable derivatives may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

Compounds of formula (I) and their pharmaceutically acceptable derivatives may also be useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

Compounds of formula (I) and their pharmaceutically acceptable derivatives may also be useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

In one embodiment compounds of the invention may bind selectively to the CB2 receptor; such compounds may be particularly useful in treating CB2 receptor mediated diseases.

The term “treatment” or “treating” as used herein includes the treatment of established disorders and also includes the prophylaxis thereof. The term “prophylaxis” is used herein to mean preventing symptoms in an already afflicted subject or preventing recurrence of symptoms in an afflicted subject and is not limited to complete prevention of an affliction.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the activity of cannabinoid 2 receptors.

According to a further aspect of the invention we provide the use of a compound of formula (I) or a pharmaceutically acceptable derivative thereof for the manufacture of a therapeutic agent for the treatment of a condition which is mediated by cannabinoid 2 receptors.

According to a further aspect of the invention, we provide a method of treating a mammal, for example a human suffering from a condition which is mediated by the activity of cannabinoid 2 receptors which comprises administering to said subject a therapeutically effective, non-toxic amount of a compound of formula (I) or a pharmaceutically acceptable derivative thereof.

According to a further aspect of the invention we provide a method of treating a mammal, for example a human suffering from an immune disorder, an inflammatory disorder, pain, rheumatoid arthritis, multiple sclerosis, osteoarthritis or osteoporosis which method comprises administering to said subject a therapeutically effective, non-toxic amount of a compound of formula (I) or a pharmaceutically acceptable derivative thereof.

In one embodiment the pain is selected from inflammatory pain, visceral pain, cancer pain, neuropathic pain, lower back pain, muscular skeletal, post operative pain, acute pain and migraine. For example, the inflammatory pain is pain associated with rheumatoid arthritis or osteoarthritis.

According to another aspect of the invention is provided the use of a compound of formula (I) or a pharmaceutically acceptable derivative thereof for the manufacture of a therapeutic agent for the treatment or prevention of a condition such as an immune disorder, an inflammatory disorder, pain, rheumatoid arthritis, multiple sclerosis, osteoarthritis or osteoporosis.

In order to use a compound of formula (I) or a pharmaceutically acceptable derivative thereof for the treatment of humans and other mammals it is normally formulated in accordance with standard pharmaceutical practice as a pharmaceutical composition. Therefore in another aspect of the invention there is provided a pharmaceutical composition comprising a compound of formula (I) or a pharmaceutically acceptable derivative thereof adapted for use in human or veterinary medicine. In one embodiment the pharmaceutical composition further comprises a pharmaceutical carrier or diluent thereof.

As used herein, “modulator” means both antagonist, partial or full agonist and inverse agonist. In one embodiment the present modulators are agonists. In another embodiment the present modulators are antagonists. In one embodiment the compounds of the invention are CB2 agonists.

Compounds of formula (I) and their pharmaceutically acceptable derivatives may be administered in a standard manner for the treatment of the indicated diseases, for example orally, parentarally, sub-lingually, dermally, intranasally, transdermally, rectally, via inhalation or via buccal administration.

Compounds of formula (I) and their pharmaceutically acceptable derivatives which are active when given orally can be formulated as liquids, tablets, capsules and lozenges. A liquid formulation will generally consist of a suspension or solution of the compound or salt in a liquid carrier for example, ethanol, olive oil, glycerine, glucose (syrup) or water with a flavouring, suspending, or colouring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers or a semi solid e.g. mono di-glycerides of capric acid, Gelucire™ and Labrasol™, or a hard capsule shell e.g gelatin. Where the composition is in the form of a soft shell capsule e.g. gelatin, any pharmaceutical carrier routinely used for preparing dispersions or suspensions may be considered, for example aqueous gums or oils, and are incorporated in a soft capsule shell.

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of formula (I) or a pharmaceutically acceptable derivative thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

In one embodiment the composition is in unit dosage form, for example a tablet, capsule or metered aerosol dose, so that the patient may administer a single dose.

Each dosage unit for oral administration contains suitably from 0.001 mg to 500 mg, for example 0.01 mg to 500 mg such as from 0.01 mg to 100 mg, and each dosage unit for parenteral administration contains suitably from 0.001 mg to 100 mg, of a compound of formula (I) or a pharmaceutically acceptable derivative thereof calculated as the free acid (underivatized compound). Each dosage unit for suppository administration contains suitably from 0.001 mg to 500 mg, for example 0.01 mg to 500 mg such as from 0.01 mg to 100 mg. Each dosage unit for intranasal administration contains suitably 1-400 mg and suitably 10 to 200 mg per person. A topical formulation contains suitably 0.01 to 5.0% of a compound of formula (I).

The daily dosage regimen for oral administration is suitably about 0.01 mg/Kg to 1000 mg/Kg, of a compound of formula (I) or a pharmaceutically acceptable derivative thereof calculated as the free acid (underivatized compound). The daily dosage regimen for parenteral administration is suitably about 0.001 mg/Kg to 200 mg/Kg, of a compound of formula (I) or a pharmaceutically acceptable derivative thereof calculated as the free acid (underivatized compound). The daily dosage regimen for suppository administration is suitably about 0.01 mg/Kg to 1000 mg/Kg, of a compound of formula (I) or a pharmaceutically acceptable derivative thereof calculated as the free acid (underivatized compound). The daily dosage regimen for intranasal administration and oral inhalation is suitably about 10 to about 500 mg/person. The active ingredient may be administered from 1 to 6 times a day, sufficient to exhibit the desired activity.

It may be advantageous to prepare the compounds of the present invention as nanoparticles. This may improve the oral bioavailability of the compounds. For the purposes of the present invention “nanoparticulate” is defined as solid particles with 50% of the particles having a particle size of less than 1 μm, for example less than 0.75 μm

The particle size of the solid particles of compound (I) may be determined by laser diffraction. A suitable machine for determining particle size by laser diffraction is a Lecotrac laser particle size analyser, using an HELOS optical bench fitted with a QUIXEL dispersion unit.

Numerous processes for the synthesis of solid particles in nanoparticulate form are known. Typically these processes involve a milling process, for example a wet milling process in the presence of a surface modifying agent that inhibits aggregation and/or crystal growth of the nanoparticles once created. Alternatively these processes may involve a precipitation process, for example, a process of precipitation in an aqueous medium from a solution of the drug in a non-aqueous solvent.

Accordingly, in a further aspect, the present invention provides a process for preparing compounds of formula (I) and their pharmaceutically acceptable derivatives in nanoparticulate form as hereinbefore defined, which process comprises milling or precipitation.

Representative processes for the preparation of solid particles in nanoparticulate form are described in the patents and publications listed below.

U.S. Pat. No. 4,826,689 to Violanto & Fischer, U.S. Pat. No. 5,145,684 to Liversidge et al
U.S. Pat. No. 5,298,262 to Na & Rajagopalan, U.S. Pat. No. 5,302,401 Liversidge et al
U.S. Pat. No. 5,336,507 to Na & Rajagopalan, U.S. Pat. No. 5,340,564 to Illig & Sarpotdar
U.S. Pat. No. 5,346,702 to Na Rajagopalan, U.S. Pat. No. 5,352,459 to Hollister et al U.S. Pat. No. 5,354,560 to Lovrecich, U.S. Pat. No. 5,384,124 to Courteille et al, U.S. Pat. No. 5,429,824 to June, U.S. Pat. No. 5,503,723 to Ruddy et al, U.S. Pat. No. 5,510,118 to Bosch et al, U.S. Pat. No. 5,518 to Bruno et al, U.S. Pat. No. 5,518,738 to Eickhoff et al, U.S. Pat. No. 5,534,270 to De Castro, U.S. Pat. No. 5,536,508 to Canal et al, U.S. Pat. No. 5,552,160 to Liversidge et al, U.S. Pat. No. 5,560,931 to Eickhoff et al, U.S. Pat. No. 5,560,932 to Bagchi et al, U.S. Pat. No. 5,565,188 to Wong et al, U.S. Pat. No. 5,571,536 to Eickhoff et al, U.S. Pat. No. 5,573,783 to Desieno & Stetsko, U.S. Pat. No. 5,580,579 to Ruddy et al, U.S. Pat. No. 5,585,108 to Ruddy et al, U.S. Pat. No. 5,587,143 to Wong, U.S. Pat. No. 5,591,456 to Franson et al, U.S. Pat. No. 5,622,938 to Wong, U.S. Pat. No. 5,662,883 to Bagchi et al, U.S. Pat. No. 5,665,331 to Bagchi et al, U.S. Pat. No. 5,718,919 to Ruddy et al, U.S. Pat. No. 5,747,001 to Wiedmann et al, WO93/25190, WO96/24336, WO 97/14407, WO 98/35666, WO 99/65469, WO 00/18374, WO 00/27369, WO 00/30615 and WO 01/41760.

Such processes may be readily adapted for the preparation of compounds of formula (I) and their pharmaceutically acceptable derivatives in nanoparticulate form. Such processes form a further aspect of the invention.

The process of the present invention may use a wet milling step carried out in a mill such as a dispersion mill in order to produce a nanoparticulate form of the compound. The present invention may be put into practice using a conventional wet milling technique, such as that described in Lachman et al., The Theory and Practice of Industrial Pharmacy, Chapter 2, “Milling” p. 45 (1986).

In a further refinement, WO02/00196 (SmithKline Beecham plc) describes a wet milling procedure using a mill in which at least some of the surfaces are made of nylon (polyamide) comprising one or more internal lubricants, for use in the preparation of solid particles of a drug substance in nanoparticulate form.

In another aspect the present invention provides a process for preparing compounds of the invention in nanoparticulate form comprising wet milling a suspension of compound in a mill having at least one chamber and agitation means, said chamber(s) and/or said agitation means comprising a lubricated nylon, as described in WO02/00196.

The suspension of a compound of the invention for use in the wet milling is typically a liquid suspension of the coarse compound in a liquid medium. By “suspension” is meant that the compound is essentially insoluble in the liquid medium. Representative liquid media include an aqueous medium. Using the process of the present invention the average particle size of coarse compound of the invention may be up to 1 mm in diameter. This advantageously avoids the need to pre-process the compound.

In a further aspect of the invention the aqueous medium to be subjected to the milling comprises compound of formula (I) or a pharmaceutically acceptable derivative thereof present in from about 1% to about 40% w/w, suitably from about 10% to about 30% w/w, for example about 20% w/w.

The aqueous medium may further comprise one or more pharmaceutically acceptable water-soluble carriers which are suitable for steric stabilisation and the subsequent processing of compound of formula (I) or pharmaceutically acceptable derivative thereof after milling to a pharmaceutical composition, e.g. by spray drying. Pharmaceutically acceptable excipients most suitable for steric stabilisation and spray-drying are surfactants such as poloxamers, sodium lauryl sulphate and polysorbates etc; stabilisers such as celluloses e.g. hydroxypropylmethyl cellulose; and carriers such as carbohydrates e.g. mannitol.

In a further aspect of the invention the aqueous medium to be subjected to the milling may further comprise hydroxypropylmethyl cellulose (HPMC) present from about 0.1 to about 10% w/w.

The process of the present invention may comprise the subsequent step of drying compound of the invention to yield a powder.

Accordingly, in a further aspect, the present invention provides a process for preparing a pharmaceutical composition containing a compound of the present invention which process comprises producing a compound of formula (I) or a pharmaceutically acceptable derivative thereof in nanoparticulate form optionally followed by drying to yield a powder, and optionally admixing with one or more pharmaceutically acceptable carriers.

A further aspect of the invention is a pharmaceutical composition comprising a compound of formula (I) or a pharmaceutically acceptable derivative thereof in which the compound of formula (I) or a pharmaceutically acceptable derivative thereof is present in solid particles in nanoparticulate form, in admixture with one or more pharmaceutically acceptable carriers or excipients.

By “drying” is meant the removal of any water or other liquid vehicle used during the process to keep compound of formula (I) in liquid suspension or solution. This drying step may be any process for drying known in the art, including freeze drying, spray granulation or spray drying. Of these methods spray drying is particularly preferred. All of these techniques are well known in the art. Spray drying/fluid bed granulation of milled compositions is carried out most suitably using a spray dryer such as a Mobile Minor Spray Dryer [Niro, Denmark], or a fluid bed drier, such as those manufactured by Glatt, Germany.

In a further aspect the invention provides a pharmaceutical composition as hereinbefore defined, in the form of a dried powder, obtainable by wet milling solid particles of compound of formula (I) followed by spray-drying the resultant suspension.

In one embodiment, the pharmaceutical composition as hereinbefore defined, further comprises HPMC present in less than 15% w/w, for example, in the range 0.1 to 10% w/w.

The CB2 receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as aspirin, diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP₁ receptor ligands, EP₄ receptor ligands; EP₂ receptor ligands; EP₃ receptor ligands; EP₄ antagonists; EP₂ antagonists and EP₃ antagonists; bradykinin receptor ligands and vanilloid receptor ligand, antirheumatoid arthritis drugs, for example anti TNF drugs e.g. enbrel, remicade, anti-IL-1 drugs, DMARDS e.g. leflunamide or 5HT₆ compounds. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

Suitable 5HT6 compounds for a combination suitable for the treatment of e.g Alzhemiers disease or cognitive enhancement, may be selected from SGS518 (Saegis), BGC20 761 (BTG disclosed in WO00/34242), WAY466 (Wyeth), PO4368554 (Hoffman le Roche), BVT5182 (Biovitron) and LY483518 (Lily), SB742457 (GSK) and/or compounds disclosed as Example 1 to 50 in WO03/080580.

The compound of the present invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compound of the inventions include for example ondansetron, granisetron, metoclopramide.

Suitable serotonin agonists which may be used in combination with the compound of the invention include sumatriptan, rauwolscine, yohimbine, metoclopramide.

Suitable SSRIs which may be used in combination with the compound of the invention include fluoxetine, citalopram, femoxetine, fluvoxamine, paroxetine, indalpine, sertraline, zimeldine.

Suitable SNRIs which may be used in combination with the compound of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Compounds of the present invention may used in combination with PDE4 inhibitors. The PDE4 inhibitor useful in this invention may be any compound that is known to inhibit the PDE4 enzyme or which is discovered to act in as PDE4 inhibitor, and which is only or essentially only a PDE4 inhibitor, not compounds which inhibit to a degree of exhibiting a therapeutic effect other members of the PDE family as well as PDE4. Generally it is preferred to use a PDE4 antagonist which has an IC₅₀ ratio of about 0.1 or greater as regards the IC₅₀ for the PDE4 catalytic form which binds rolipram with a high affinity divided by the IC₅₀ for the form which binds rolipram with a low affinity. Compounds of the present invention or combinations with PDE4 can be used in treating inflammation and as bronchodilators.

There are at least two binding forms on human monocyte recombinant PDE 4 (hPDE 4) at which inhibitors bind. One explanation for these observations is that hPDE 4 exists in two distinct forms. One binds the likes of rolipram and denbufylline with a high affinity while the other binds these compounds with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

Reference is made to U.S. Pat. No. 5,998,428, which describes these methods in more detail. It is incorporated herein in full as though set forth herein.

Suitably the PDE4 inhibitors are those PDE4 inhibitors which have an IC₅₀ ratio of greater than 0.5, and particularly those compounds having a ratio of greater than 1.0.

A further aspect of the invention is an CB2 modulator (a compound of formula (I) or a pharmaceutically acceptable derivative thereof) in combination with a PDE4 inhibitor and pharmaceutical compositions comprising said combination.

A further aspect of the invention is a method of treating lung disorders for example asthma, bronchitis, emphysema, allergic rhinitis, respiratory distress syndrome, pigeon fancier's disease, farmer's lung, chronic obstructive pulmonary disease, (COPD) and cough or a disorder which can be treated with a broncodilator which comprises administering to a mammal including man, an effective amount of a CB2 modulator ((a compound of formula (I) or a pharmaceutically acceptable derivative thereof) and an effective amount of a PDE4 inhibitor or a pharmaceutically acceptable derivative thereof.

An additional aspect of the invention is the use of an effective amount of a CB2 modulator (a compound of formula (I) or a pharmaceutically acceptable derivative thereof) and an effective amount of a PDE4 inhibitor or a pharmaceutically acceptable derivative thereof in the manufacture of a medicament in the treatment of lung disorders for example asthma, bronchitis, emphysema, allergic rhinitis, respiratory distress syndrome, pigeon fancier's disease, farmer's lung, chronic obstructive pulmonary disease, (COPD) and cough or for the manufacture of a brocodilator.

When used herein cough can have a number of forms and includes productive, non-productive, hyper-reactive, asthma and COPD associated.

A further aspect of the invention is a patient pack comprising an effective amount of a CB2 modulator (a compound of formula (I) or a pharmaceutically acceptable derivative thereof) and an effective amount of a PDE4 inhibitor or a pharmaceutically acceptable derivative thereof.

Possible PDE4 compounds are cis [cyano-4-(3-cyclopentyloxy-4-methoxyphenyl)cyclohexan-1-carboxylate] also known as cilomilast or Ariflo®, 2-carbomethoxy-4-cyano-4-(3-cyclopropyl methoxy-4-difluoromethoxyphenyl)cyclohexan-1-one, and cis [4-cyano-4-(3-cyclopropylmethoxy-4-difluoromethoxyphenyl)cyclohexan-1-ol]. They can be made by the processed described in U.S. Pat. Nos. 5,449,686 and 5,552,438. Other PDE4 inhibitors, specific inhibitors, which can be used in this invention are AWD-12-281 from ASTA MEDICA (Hofgen, N. et al. 15th EFMC Int Symp Med Chem (September 6-10, Edinburgh) 1998, Abst P. 98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO 99/47505) from Byk-Gulden (now Altana); or a compound identified as T-440 (Tanabe Seiyaku; Fuji, K. et al. J Pharmacol Exp Ther, 1998, 284 (1): 162).

Additional PDE4 inhibitors are disclosed on pages 2 to 15 of WO01/13953. Specifically selected are arofylline, atizoram, BAY-19-8004, benafentrine, BYK-33043, CC-3052, CDP-840, cipamfylline, CP-220629, CP-293121, D-22888, D-4396, denbufylline, filaminast, GW-3600, ibudilast, KF-17625, KS-506-G, laprafylline, NA-0226A, NA-23063A, ORG-20241, ORG-30029, PDB-093, pentoxifylline, piclamilast, rolipram, RPR-117658, RPR-122818, RPR-132294, RPR-132703, RS-17597, RS-25344-000, SB-207499, SB210667, SB211572, SB-211600, SB212066, SB212179, SDZ-ISQ-844, SDZ-MNS-949, SKF-107806, SQ-20006, T-2585, tibenelast, tolafentrine, UCB-29646, V-11294A, YM-58997, YM-976 and zardaverine.

In one embodiment the PDE4 inhibitor is selected from cilomilast, AWD-12-281, NCS-613, D-4418, CI-1018, V-11294A, roflumilast or T-440.

Compounds of the present invention may also be of use in treating atherosclerosis in combination with an anti-hyperlipidaemic, anti-atherosclerotic, anti-diabetic, anti-anginal, anti-hypertension agent or an agent for lowering Lp(a). Examples of the above include cholesterol synthesis inhibitors such as statins, anti-oxidants such as probucol, insulin sensitisers, calcium channel antagonists. Examples of agents for lowering Lp(a) include the aminophosphonates described in WO 97/02037, WO 98/28310, WO 98/28311 and WO 98/28312 (Symphar SA and SmithKline Beecham). Examples of antihyerpertension agents are angiotensin-converting enzyme inhibitors, angiotensin-II receptor antagonists, ACE/NEP inhibitors, -blockers, calcium channel blockers, PDE inhibitors, aldosterone blockers

A possible combination therapy will be the use of a compound of the present invention and a statin. The statins are a well known class of cholesterol lowering agents and include atorvastatin, simvarstatin, pravastatin, cerivastatin, fluvastatin, lovastatin and ZD 4522 (also referred to as S-4522, Astra Zeneca). The two agents may be administered at substantially the same time or at different times, according to the discretion of the physician.

A further possible combination therapy will be the use of a compound of the present invention and an anti-diabetic agent or an insulin sensitiser. Within this class, possible compounds for use with a compound of the present invention include the PPARgamma activators, for instance G1262570 (Glaxo Wellcome) and also the glitazone class of compounds such as rosiglitazone (Avandia, SmithKline Beecham), troglitazone and pioglitazone.

It will be appreciated that the compounds of any of the above combinations or compositions may be administered simultaneously (either in the same or different pharmaceutical formulations), separately or sequentially.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

### Determination of Cannabinoid CB1 Receptor Agonist Activity

The cannabinoid CB1 receptor agonist activity of compounds of formula (I) was determined in accordance with the following experimental method.

### Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB1 and a yeast transcriptional terminator sequence to the 3′ end of CB1. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20 μM fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50[compound X]}/EC_{50[HU210]}

Where EC_{50 [compound X]} is the EC₅₀ of compound X and EC_{50 [HU210]} is the EC₅₀ of HU210.

### Determination of Cannabinoid CB2 Receptor Agonist Activity

The cannabinoid CB2 receptor agonist activity of compounds of formula (I) was determined in accordance with the following experimental method.

### Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM solutions in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black microtitre plates from NUNC (384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20M fluorescein di-β-D-glucopyranoside (FDGlu). This mixture (50 ul per well) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a fluorescence microtitre plate reader (Tecan Spectrofluor or LJL Analyst excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210]}−Min_{[HU210]}×100%

where Max_{[compound X]} and Min_{[compound X]} are the fitted maximum and minimum respectively from the concentration effect curve for compound X, and Max_{[HU210]} and Min_{[HU}210] are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50[compound X]}/EC_{50[HU210]}

Where EC_{50 [compound X]} is the EC₅₀ of compound X and EC_{50 [HU210]} is the EC₅₀ of HU210.

### Results

The compounds of the Examples tested according to the above methods had an EMR of between 1 and 3000 in the CB1 yeast receptor assay and an EMR of between 0.1 and 100 in the CB2 yeast receptor assay. Compounds of Examples 2-15, 19-25, 27, 28, and 30-34 had at least a tenfold lower EMR for CB2 over CB1. The results given are averages of a number of experiments.

### Measurement of CB2 Agonist Effects in a Reporter Gene Assay

### Experimental Method

CB2 agonist effects may be determined using a reporter gene assay as follows. These studies may be performed using a CHO-K1 cell line expressing human recombinant CB2 receptors (CHO-K1 CB2 CRE-LUC cells). These cells additionally express a “CRE-LUC” reporter gene construct comprising the gene for luciferase under the control of multiple cAMP response element binding protein promoters. In these cells, increases in intracellular cAMP levels leads to transcription of the luciferase gene and the subsequent production of luciferase. The expression of luciferase is measured by addition to the cells of a proprietary mixture containing luciferin, the substrate for luciferase (Luclite, Perkin Elmer, Cat No 6016919). The resultant reaction leads to the generation of light which is measured in a TopCount scintillation counter. In the CHO-K1 CB2 CRE-LUC cells, forskolin produces a marked increase in luciferase expression and CB2 agonists inhibit this response. The CHO-K1 CB2 CRE-LUC cells routinely express a high level of constitutive CB2 receptor activity. This may be overcome in these experiments by pre-treating the cells with the inverse agonist, SR144528, for 30-60 mins before use. This treatment has been shown to eliminate constitutive CB2 receptor activity (Bouaboula et al., 1999).

### Methods

CHO-K1 CB2 CRE-LUC cells are grown in DMEM/F12 plus glutamax I medium (Gibco Cat. No. 31331-028), supplemented with 9% FBS (Gibco, Cat. No. 16000-040) and 0.5 mg.ml⁻¹ G418 (Gibco, Cat. No. 10131-027) and 0.5 mg.ml⁻¹ Hygromycin (Invitrogen, Cat. No. 10687-010). Cells are grown as a monolayer culture in 162 cm² vented Nunclon flasks (NUNC, Cat. No. 178883) in 27.5 ml of media in a humidified 95% air and 5% CO₂ atmosphere at 37° C. When confluent, the growth media is replaced with DMEM/F12 medium (Gibco, Cat. No. 31331-028) containing 100 nM of the CB2 inverse agonist, SR144528, and the cells are incubated at 37° C. for 30-60 mins. Flasks are rinsed twice with 25 ml Dulbecco's phosphate buffered saline (PBS, Gibco Cat. No. 14190-094) and then harvested by incubation for 10 mins in 10 ml of Versene (Gibco, Cat. No. 15040-033). Cells are detached by a sharp blow to the flask and the cell suspension made up to 50 ml with PBS and centrifuged at 250×g for 5 mins. The cell pellet is re-suspended in 24 mls of phenol-red free DMEM/F12 assay buffer (Gibco, Cat. No. 11039-021) and 50 μl of cell suspension (approximately 50,000 cells) added to 96 well plates (Costar, Cat. No. 3904-clear bottomed black well plates) containing 50 μl of test agonist in 2 μM forskolin (final assay concentration of 1 μM FSK). Test agonists are prepared as 10 mM solutions in DMSO and diluted into phenol-red free DMEM/F12 assay buffer containing 2 μM forskolin to produce a 20 μM solution of test agonist. Subsequent serial dilutions of test agonist are prepared in the assay buffer containing forskolin and each test agonist is routinely examined over a final assay concentration range of 10 μM to 10 nM (or lower if required). The plates are mixed on a plate shaker for 5 mins (800-1000 rpm) and then centrifuged briefly (5-10 s) at 250×g, placed in a Bioplate without their lids, and incubated for 4-5 hr in a humidified 95% air and 5% CO₂ atmosphere at 37° C. The 96 well plates are removed from the incubator and placed at RT for 10-15 mins before addition of 25 μl of Luclite solution, prepared according to the manufacturer's instructions. The plates are sealed with Topseal A (Perkin Elmer, Cat. No. 6005185), mixed on a plate shaker for 5 mins (800-1000 rpm) and then centrifuged briefly (5-10 s) at 250×g. Finally, luminescence is measured using a Packard TopCount scintillation counter.

### Data Analysis

For each compound maximal inhibition of the forsklin response and the EC50 for this effect is determined. In each experiment the reference agonist HU210 is included and the maximal effect of each test agonist is expressed relative to the maximal effect produced by HU210 to provide an estimate of intrinsic activity. In addition the EC50 of each compound is divided by the EC50 for HU210 to calculate the equipotent molar ratio (EMR) for the test compound.

### REFERENCE

Bouaboula M. Dussossoy D. Casellas P. Regulation of peripheral cannabinoid receptor CB2 phosphorylation by the inverse agonist SR 144528. Implications for receptor biological responses. Journal of Biological Chemistry. 274 (29):20397-405, 1999

The following examples are illustrative, but not limiting of the embodiments of the present invention.

### ABBREVIATIONS

LC/MS (Liquid chromatography/Mass spectroscopy), MDAP (Mass Directed AutoPurification), NMR (Nuclear Magnetic Resonance)

### MDAP System

### Hardware

### Waters 2525 Binary Gradient Module

### Waters 515 Makeup Pump

### Waters Pump Control Module

### Waters 2767 Inject Collect

### Waters Column Fluidics Manager

### Waters 2996 Photodiode Array Detector

### Waters ZQ Mass Spectrometer

Gilson 202 fraction collector
Gilson Aspec waste collector

### Software

Waters Masslynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol:Water 80:20
Needle rinse solvent=Methanol

### Methods

There are four methods used depending on the analytical retention time of the compound of interest. They all have a 13.5-minute runtime, which comprises of a 10-minute gradient followed by a 3.5 minute column flush and re-equilibration step.

### Large/Small Scale 1.0-1.5=5-30% B

### Large/Small Scale 1.5-2.2=15-55% B

### Large/Small Scale 2.2-2.9=30-85% B

### Large/Small Scale 2.9-3.6=50-99% B

Large/Small Scale 3.6-5.0=80-99% B (in 6 minutes)

### Flow Rate

All of the above methods have a flow rate of either 20 mls/min (Small Scale) or 40 mls/min (Large Scale)

### Analytical LCMS Systems

### Hardware

### Agilent 1100 Gradient Pump

### Agilent 1100 Autosampler

### Agilent 1100 DAD Detector

### Agilent 1100 Degasser

### Agilent 1100 Oven

### Agilent 1100 Controller

### Waters ZQ Mass Spectrometer

### Sedere Sedex 75 or Sedere Sedex 85 or Polymer Labs PL-ELS-2100

### Software

Waters MassLynx version 4.0 SP2

### Column

The column used is a Waters Atlantis, the dimensions of which are 4.6 mm×50 mm. The stationary phase particle size is 3 μm.

### Solvents

A: Aqueous solvent=Water+0.05% Formic Acid
B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

### Flow Rate

The above method has a flow rate of 3 ml/mins

### Conditions Used for NMR

### Hardware

### Bruker 400 MHz Ultrashield

### Bruker B-ACS60 Autosampler

### Bruker Advance 400 Console

### Software

User interface—NMR Kiosk
Controlling software—XWin NMR version 3.0

### Conditions Used for the Microwave

### Hardware

### Biotage Initiator

### Specifications

Heating temperature up to 250° C.
Microwave radiation 50-300 W at 2.45 GHz

### Intermediate 1

### Ethyl 1-[2-(ethyloxy)-2-oxoethyl]-5-methyl-1H-pyrrole-2-carboxylate

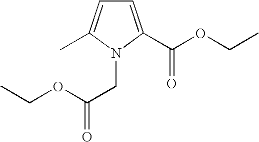

To a solution of ethyl 5-methyl-1H-pyrrole-2-carboxylate (prepared by the method of: Curran, Timothy P.; Keaney, Meghan T. Journal of Organic Chemistry (1996), 61 (25), 9068-9069) (4.08 g) in dry N,N-dimethylformamide (32 ml) at 0° C. under argon was added sodium hydride (60% dispersion in mineral oil, 1.28 g). After effervescence had ceased (fifteen minutes), a solution of ethyl bromoacetate (3.3 ml) in dry N,N-dimethylformamide (8 ml) was added dropwise and the reaction mixture allowed to warm to room temperature. The reaction mixture was diluted with ethyl acetate (400 ml), washed twice with water (2×100 ml) then dried (MgSO₄), and evaporated to afford the title compound as an orange oil (8.4 g).

LC/MS [MH⁺] 240 consistent with molecular formula C₁₂H₁₇NO₄.

### Intermediate 2

### Ethyl 1-{(E)-1-[(ethyloxy)carbonyl]-2-hydroxyethenyl}-5-methyl-1H-pyrrole-2-carboxylate

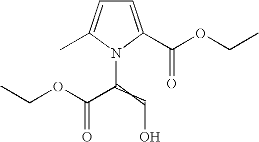

To a solution of ethyl 1-[2-(ethyloxy)-2-oxoethyl]-5-methyl-1H-pyrrole-2-carboxylate (8.4 g) in dry tetrahydrofuran at 0° C. under argon was added, portionwise, sodium hydride (60% dispersion in mineral oil, 2.67 g). After thirty minutes, ethyl formate (9.8 ml) was added dropwise and the iced water bath removed. The reaction mixture was warmed to initiate the reaction, and once initiated, cooled in an iced water bath to control the observed exotherm. When the exotherm had subsided, the iced water bath was removed and the reaction mixture was stirred at room temperature for one hour and thirty minutes. Ethyl formate (2 ml, 2 ml and 4 ml) was added at one hour intervals and stirring continued overnight. The reaction mixture was quenched with ethanol and evaporated. The residue was dissolved in saturated ammonium chloride (100 ml) and acidified with 5N hydrochloric acid to pH1. The aqueous mixture was extracted with ethyl acetate (200 ml) and the organic layer washed twice with water (2×100 ml). The organic layer was dried (MgSO₄), and evaporated to afford a brown oil. The oil was dissolved in dichloromethane and applied to a Biotage column (100 g silica) and the column eluted with hexane, 25% ethyl acetate/hexane and 50% ethyl acetate/hexane to afford the title compound as an orange oil (6.65 g).

LC/MS [MH⁺] 268 consistent with molecular formula C₁₃H₁₇NO₅.

### Intermediate 3

### Ethyl 1-{(E)-2-amino-1-[(ethyloxy)carbonyl]ethenyl}-5-methyl-1H-pyrrole-2-carboxylate

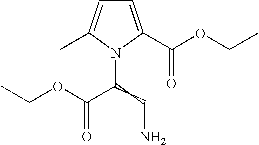

A solution of ethyl 1-{(E)-1-[(ethyloxy)carbonyl]-2-hydroxyethenyl}-5-methyl-1H-pyrrole-2-carboxylate (6.65 g) and ammonium acetate (9.6 g) in ethanol (80 ml) was refluxed for two hours. The ethanol was evaporated and the residue partitioned between ethyl acetate (200 ml) and water (100 ml). The organic layer was dried (MgSO₄), and evaporated to afford the title compound as an orange oil (7.29 g).

LC/MS [MH⁺] 267 consistent with molecular formula C₁₃H₁₈N₂O₄.

### Intermediate 4

### Ethyl 6-methyl-1-oxo-1,2-dihydropyrrolo[1,2-a]pyrazine-4-carboxylate

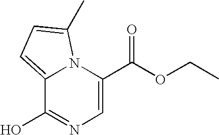

A mixture of ethyl 1-{(E)-2-amino-1[(ethyloxy)carbonyl]ethenyl}-5-methyl-1H-pyrrole-2-carboxylate (200 mg) and sodium tert-butoxide (72 mg) in N,N-dimethylformamide (3 ml) was heated a 160° C. under microwave conditions for thirty minutes. A solution of ethyl 1-{(E)-2-amino-1-[(ethyloxy)carbonyl]ethenyl}-5-methyl-1H-pyrrole-2-carboxylate (200 mg)) in N,N-dimethylformamide (3 ml) was treated with sodium hydride (60% dispersion in mineral oil, 36 mg) and after fifteen minutes was heated at 160° C. under microwave conditions for thirty minutes. A mixture of ethyl 1-{(E)-2-amino-1-[(ethyloxy)carbonyl]ethenyl}-5-methyl-1H-pyrrole-2-carboxylate (1.37 g) and sodium tert-butoxide (495 mg) in N,N-dimethylformamide (15 ml) was heated at 160° C. under microwave conditions for thirty minutes. The reaction mixtures above were added to iced water and extracted twice with ethyl acetate (2×50 ml). The combined ethyl acetate layers were washed with water (40 ml) then dried (MgSO₄), and evaporated to afford a pale orange solid. The solid was triturated with diethyl ether, refrigerated overnight, filtered off, washed with ice cold diethyl ether and dried to afford the title compound as a white solid (542 mg).

LC/MS [MH⁺] 221 consistent with molecular formula C₁₁H₁₂N₂O₃.

### Intermediate 5

### Ethyl 1-chloro-6-methylpyrrolo[1,2-a]pyrazine-4-carboxylate

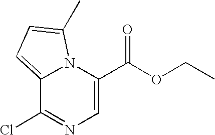

A suspension of ethyl 6-methyl-1-oxo-1,2-dihydropyrrolo[1,2-a]pyrazine-4-carboxylate (536 mg) in phenyl dichlorophosphate (4 ml) was heated at 170° C. to give a brown solution. The solution was allowed to cool and then poured onto iced water. The aqueous mixture was neutralised by careful addition of solid sodium hydrogen carbonate and then extracted with ethyl acetate. The organic layer was washed with 5% aqueous sodium hydrogen carbonate and water and then dried (MgSO₄) and evaporated to afford the title compound as a brown oil (694 mg).

LC/MS [MH⁺] 239 consistent with molecular formula C₁₁H₁₁³⁵ClN₂O₂.

### Intermediate 6

### 1-Chloro-6-methylpyrrolo[1,2-a]pyrazine-4-carboxylic acid

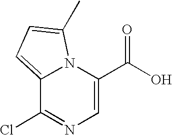

Solutions of ethyl 1-chloro-6-methylpyrrolo[1,2-a]pyrazine-4-carboxylate (114 mg and 580 mg) in ethanol (1 ml and 6 ml) were treated with 2N sodium hydroxide (1 ml and 6 ml) and stirred at room temperature for one hour. The reaction mixtures were combined and evaporated. The residue was dissolved in water and washed with diethyl ether. The aqueous layer was acidified with 5N hydrochloric acid to pH1 and extracted with ethyl acetate. The organic layer was washed twice with water then dried (MgSO₄) and evaporated to afford the title compound as a pale yellow solid (470 mg).

LC/MS [MH⁺] 211 consistent with molecular formula C₉H₇³⁵ClN₂O₂.

### Intermediate 7

### 1-Chloro-6-methyl-4-(4-morpholinylcarbonyl)pyrrolo[1,2-a]pyrazine

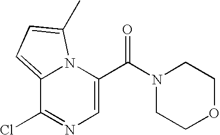

To a solution of 1-chloro-6-methylpyrrolo[1,2-a]pyrazine-4-carboxylic acid (150 mg) in dry N,N-dimethylformamide (6 ml) was added N-ethyl diisopropylamine (621 ul), morpholine (124 ul) and O-(1H-benzotriazol-1-yl)-N,N,N′,N′-tetramethyluronium hexafluorophosphate (407 mg) and the reaction mixture stirred at room temperature for one hour. The reaction mixture was diluted with ethyl acetate and washed twice with saturated aqueous sodium hydrogen carbonate and with water and then dried (MgSO₄) and evaporated to afford the title compound as a pale yellow solid (205 mg).

LC/MS [MH⁺] 280 consistent with molecular formula C₁₃H₁₄³⁵ClN₃O₂.

### Intermediate 8

### 1-Chloro-6-methyl-N-(tetrahydro-2H-pyran-4-ylmethyl)pyrrolo[1,2-a]pyrazine-4-carboxamide

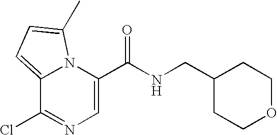

To a solution of 1-chloro-6-methylpyrrolo[1,2-a]pyrazine-4-carboxylic acid (150 mg) in dry N,N-dimethylformamide (6 ml) was added N-ethyl diisopropylamine (621 ul), (tetrahydro-2H-pyran-4-ylmethyl)amine hydrochloride (215 mg) and O-(1H-benzotriazol-1-yl)-N,N,N′,N′-tetramethyluronium hexafluorophosphate (407 mg) and the reaction mixture stirred at room temperature for two hours. N-ethyl diisopropylamine (250 ul) was added and the solution stirred at room temperature overnight. N-ethyl diisopropylamine (621 ul), (tetrahydro-2H-pyran-4-ylmethyl)amine hydrochloride (430 mg) and O-(1H-benzotriazol-1-yl)-N,N,N′,N′-tetramethyluronium hexafluorophosphate (407 mg) were added and the reaction mixture stirred at room temperature for five hours. The reaction mixture was diluted with ethyl acetate and washed twice with saturated aqueous sodium hydrogen carbonate and with water and then dried (MgSO₄) and evaporated to afford an orange oil. Purification by MDAP afforded the title compound as a yellow foam (90 mg).

LC/MS [MH⁺] 308 consistent with molecular formula C₁₅H₁₈³⁵ClN₃O₂.

### Intermediate 8(a)

### 1-Chloro-6-methyl-N-(tetrahydro-2H-pyran-4-ylmethyl)pyrrolo[1,2-a]pyrazine-4-carboxamide

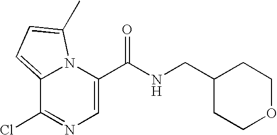

To a solution of 1-chloro-6-methylpyrrolo[1,2-a]pyrazine-4-carboxylic acid (400 mg) in dry N,N-dimethylformamide (4 ml) was added N-ethylmorpholine (1.45 ml), (tetrahydro-2H-pyran-4-ylmethyl)amine hydrochloride (575 mg), 1-hydroxybenzotriazole hydrate (402 mg) and N-(3-dimethylaminopropyl)-N′-ethylcarbodiimide (438 mg) and the solution stirred at room temperature overnight. The reaction mixture was diluted with ethyl acetate (100 ml) and washed with 1:1 saturated aqueous sodium hydrogen carbonate:water (50 ml) and with water (50 ml) and then dried (MgSO₄) and evaporated. The residue was dissolved in dichloromethane and applied to a Biotage column (10 g silica) and the column eluted with 60-100% ethyl acetate/hexane to afford the title compound as a yellow foam (240 mg).

LC/MS [MH⁺] 308 consistent with molecular formula C₁₅H₁₈³⁵ClN₃O₂.

Intermediate 8(a) contained 1-(1H-1,2,3-benzotriazol-1-yloxy)-6-methyl-N-(tetrahydro-2H-pyran-4-ylmethyl)pyrrolo[1,2-a]pyrazine-4-carboxamide (17% by LCMS)

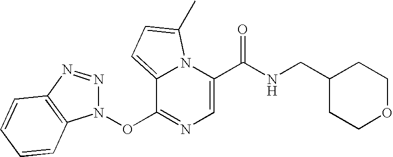

LC/MS [MH⁺] 407 consistent with molecular formula C₂₁H₂₂N₆O₃.

### Intermediate 8(b)

### 1-Chloro-6-methyl-N-(tetrahydro-2H-pyran-4-ylmethyl)pyrrolo[1,2-a]pyrazine-4-carboxamide

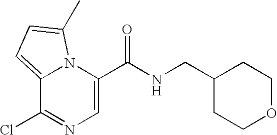

To a solution of 1-chloro-6-methylpyrrolo[1,2-a]pyrazine-4-carboxylic acid (2 g) in dry N,N-dimethylformamide (20 ml) was added N-ethylmorpholine (7.25 ml), (tetrahydro-2H-pyran-4-ylmethyl)amine hydrochloride (2.875 g), 1-hydroxybenzotriazole hydrate (2.01 g) and N-(3-dimethylaminopropyl)-N′-ethylcarbodiimide (2.19 g) and the solution stirred at room temperature over the weekend. The reaction mixture was diluted with ethyl acetate (500 ml) and washed with 1:1 saturated aqueous sodium hydrogen carbonate: water (250 ml) and with water (250 ml) and then dried (MgSO₄) and evaporated to afford the title compound as a red oil (3.7 g).

LC/MS [MH⁺] 308 consistent with molecular formula C₁₅H₁₈³⁵ClN₃O₂.

Intermediate 8(b) contained 1-(1H-1,2,3-benzotriazol-1-yloxy)-6-methyl-N-(tetrahydro-2H-pyran-4-ylmethyl)pyrrolo[1,2-a]pyrazine-4-carboxamide (70% by LCMS)

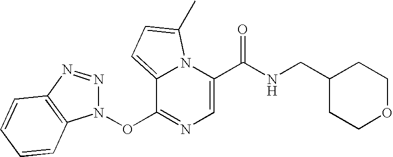

LC/MS [MH⁺] 407 consistent with molecular formula C₂₁H₂₂N₆O₃.

### Intermediate 9

### 1-Chloro-6-methyl-N-[2-(methyloxy)ethyl]pyrrolo[1,2-a]pyrazine-4-carboxamide

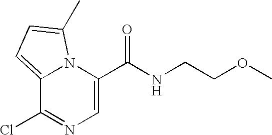

To a solution of 1-chloro-6-methylpyrrolo[1,2-a]pyrazine-4-carboxylic acid (400 mg) in dry N,N-dimethylformamide (4 ml) was added N-ethylmorpholine (968 ul), 2-methoxyethylamine (330 ul), 1-hydroxybenzotriazole hydrate (402 mg) and N-(3-dimethylaminopropyl)-N′-ethylcarbodiimide (438 mg) and the solution stirred at room temperature overnight. The reaction mixture was diluted with ethyl acetate (100 ml) and washed with 1:1 saturated aqueous sodium hydrogen carbonate:water (50 ml) and with water (50 ml) and then dried (MgSO₄) and evaporated. The oil was dissolved in dichloromethane and applied to a Biotage column (10 g silica) and the column eluted with 60-100% ethyl acetate/hexane to afford the title compound as a yellow oil (164 mg).

LC/MS [MH⁺] 268 consistent with molecular formula C₁₂H₁₄³⁵ClN₃O₂.

Intermediate 9 contained 1-(1H-1,2,3-benzotriazol-1-yloxy)-6-methyl-N-[2-(methyloxy)ethyl]pyrrolo[1,2-a]pyrazine-4-carboxamide (15% by LCMS)

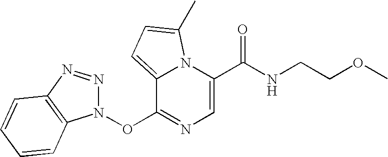

LC/MS [MH⁺] 367 consistent with molecular formula C₁₈H₁₈N₆O₃.

### Intermediate 10

### 5-chloro-7-nitro-2,3-dihydro-1-benzofuran

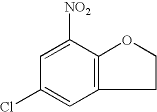

To a mixture of 5-chloro-2,3-dihydro-1-benzofuran (prepared by the method of: Ramon J. Alabaster, Ian F. Cottrell, Hugh Marley, Stanley H. B. Wright Synthesis (1988), (12), 950-952) (4.3 g) in glacial acetic acid (20 ml) and concentrated sulphuric acid (4 ml) at 100° C. was added concentrated nitric acid (1.57 ml) and glacial acetic acid (1 ml). After forty five minutes, the mixture was allowed to cool and dissolved in dichloromethane (250 ml). The organic layer was washed with water (250 ml), 1M ammonium hydroxide (250 ml) and water (250 ml) and then dried (MgSO₄) and evaporated. The solid was dissolved in dichloromethane and applied to a column and the column eluted to afford the title compound as a yellow solid (3.4 g).

### Intermediate 11

### 5-chloro-2,3-dihydro-1-benzofuran-7-amine

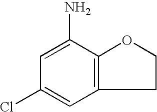

To a hot solution of 5-chloro-7-nitro-2,3-dihydro-1-benzofuran (3.7 g) in methanol (65 ml) containing iron powder (reduced) (2.7 g) was added saturated ammonium chloride solution (130 ml). The mixture was stirred under reflux for two hours then cooled and filtered over Celite. The residue was washed with a mixture of dichloromethane and methanol then dichloromethane and the combined organic layers were separated from the aqueous. The aqueous layer was extracted twice with dichloromethane and the extracts combined with the organic layer above. The organic layer was dried (MgSO₄) and evaporated to afford the title compound as a solid (3 g).

### EXAMPLE 1

### N-(3-Fluorophenyl)-6-methyl-4-(4-morpholinylcarbonyl)pyrrolo[1,2-a]pyrazin-1-amine hydrochloride

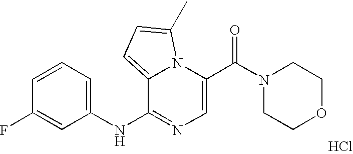

To a suspension of 1-chloro-6-methyl-4-(4-morpholinylcarbonyl)pyrrolo[1,2-a]pyrazine (67 mg) in 1,4-dioxane (1 ml) was added 3-fluoroaniline (50 ul) and methanesulfonic acid (31 ul) and the reaction mixture heated under microwave conditions at 180° C. for fifteen minutes. The solid mass was dissolved in methanol, transferred to a round bottomed flask and evaporated. The residue was partitioned between ethyl acetate (10 ml) and water (4 ml). The separated aqueous was basified with saturated aqueous sodium hydrogen carbonate (2 ml) and extracted with ethyl acetate. The organic layers were combined, dried (MgSO₄) and evaporated to a brown oil. The oil was purified by MDAP, triturated with diethyl ether then dissolved in ethyl acetate and treated with 1M hydrochloric acid in diethyl ether to afford a white solid. The mixture was evaporated and dried to afford the title compound (38 mg).

LC/MS [MH⁺] 355 consistent with molecular formula C₁₉H₁₉FN₄O₂

### EXAMPLE 2

### N-(3-Chlorophenyl)-6-methyl-4-(4-morpholinylcarbonyl)pyrrolo[1,2-a]pyrazin-1-amine hydrochloride

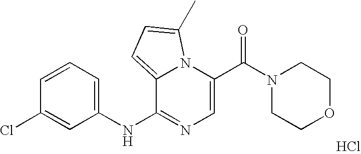

To a suspension of 1-chloro-6-methyl-4-(4-morpholinylcarbonyl)pyrrolo[1,2-a]pyrazine (67 mg) in 1,4-dioxane (1 ml) was added 3-chloroaniline (50 ul) and methanesulfonic acid (31 ul) and the reaction mixture heated under microwave conditions at 180° C. for fifteen minutes. The solid mass was dissolved in methanol, transferred to a round bottomed flask and evaporated. The residue was partitioned between ethyl acetate (10 ml), water (4 ml) and saturated aqueous sodium hydrogen carbonate (2 ml) and washed. The organic layer was dried (MgSO₄) and evaporated to a brown oil. The oil was purified by MDAP, triturated with diethyl ether/hexane, dried, then dissolved in ethyl acetate and treated with 1M hydrochloric acid in diethyl ether to afford a white solid. The mixture was evaporated and dried to afford the title compound (38 mg).

LC/MS [MH⁺] 355 consistent with molecular formula C₁₉H₁₉³⁵ClN₄O₂

### EXAMPLE 3

### N-(3-Bromophenyl)-6-methyl-4-(4-morpholinylcarbonyl)pyrrolo[1,2-a]pyrazin-1-amine hydrochloride

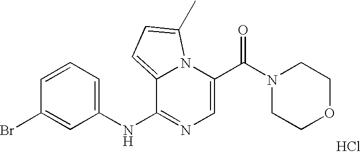

Prepared in a manner similar to Example 2 from 1-chloro-6-methyl-4-(4-morpholinylcarbonyl)pyrrolo[1,2-a]pyrazine (67 mg) (Intermediate 7) and 3-bromoaniline (52 ul) to afford the title compound as a white solid (43 mg).

LC/MS [MH⁺] 417 consistent with molecular formula C₁₉H₁₉⁸¹BrN₄O₂

### EXAMPLE 4

### 6-Methyl-4-(4-morpholinylcarbonyl)-N-{3-[(trifluoromethyl)oxy]phenyl}pyrrolo[1,2-a]pyrazin-1-amine hydrochloride

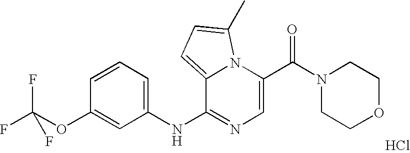

To a suspension of 1-chloro-6-methyl-4-(4-morpholinylcarbonyl)pyrrolo[1,2-a]pyrazine (100 mg) in 1,4-dioxane (1 ml) was added 3-(trifluoromethoxy)aniline (96 ul) and methanesulfonic acid (46 ul) and the reaction mixture heated under microwave conditions at 180° C. for fifteen minutes. The solid mass was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a yellow solid. The solid was dissolved in dichloromethane, applied to a Biotage Si 25+S column and eluted with 10% ethyl acetate/hexane followed by 20% and 40% ethyl acetate/hexane to afford a white solid. The solid was dissolved in ethyl acetate and treated with 1M hydrochloric acid in diethyl ether. The solution was evaporated and the residue triturated with diethyl ether, the solid filtered off and dried to afford the title compound (113 mg).

LC/MS [MH⁺] 421 consistent with molecular formula C₂₀H₁₉F₃N₄O₃

### EXAMPLE 5

### N-[2-Fluoro-3-(trifluoromethyl)phenyl]-6-methyl-4-(4-morpholinylcarbonyl)pyrrolo[1,2-a]pyrazin-1-amine hydrochloride

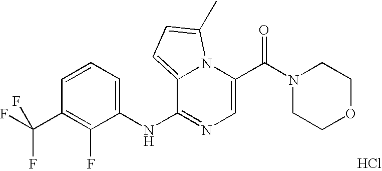

Prepared in a manner similar to Example 4 from 1-chloro-6-methyl-4-(4-morpholinylcarbonyl)pyrrolo[1,2-a]pyrazine (100 mg) (Intermediate 7) and 2-fluoro-3-(trifluoromethyl)aniline (92 ul) to afford the title compound as a white solid (88 mg).

LC/MS [MH⁺] 423 consistent with molecular formula C₂₀H₁₈F₄N₄O₂

### EXAMPLE 6

### 6-Methyl-N-[2-methyl-3-(trifluoromethyl)phenyl]-4-(4-morpholinylcarbonyl)pyrrolo[1,2-a]pyrazin-1-amine hydrochloride

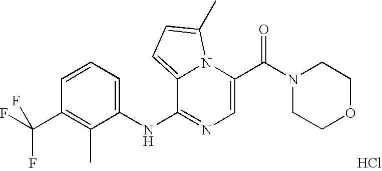

Prepared in a manner similar to Example 4 from 1-chloro-6-methyl-4-(4-morpholinylcarbonyl)pyrrolo[1,2-a]pyrazine (100 mg) (Intermediate 7) and 3-amino-2-methylbenzotrifluoride (125 mg) to afford the title compound as a white solid (64 mg).

LC/MS [MH⁺] 419 consistent with molecular formula C₂₁H₂₁F₃N₄O₂

### EXAMPLE 7

### N-[5-fluoro-2-(methyloxy)phenyl]-6-methyl-4-(4-morpholinylcarbonyl)pyrrolo[1,2-a]pyrazin-1-amine hydrochloride

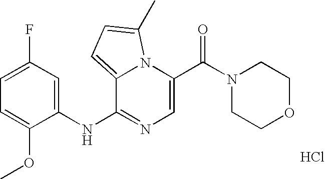

Prepared in a manner similar to Example 4 from 1-chloro-6-methyl-4-(4-morpholinylcarbonyl)pyrrolo[1,2-a]pyrazine (100 mg) (Intermediate 7) and 5-fluoro-2-methoxyaniline (100 mg) to afford the title compound as a white solid (99 mg).

LC/MS [MH⁺] 385 consistent with molecular formula C₂₀H₂₁FN₄O₃

### EXAMPLE 8

### N-(3-Fluoro-4-methylphenyl)-6-methyl-4-(4-morpholinylcarbonyl)pyrrolo[1,2-a]pyrazin-1-amine hydrochloride

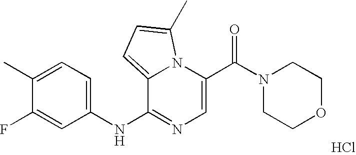

Prepared in a manner similar to Example 4 from 1-chloro-6-methyl-4-(4-morpholinylcarbonyl)pyrrolo[1,2-a]pyrazine (100 mg) (Intermediate 7) and 3-fluoro-4-methylaniline (91 mg) to afford the title compound as a white solid (118 mg).

LC/MS [MH⁺] 369 consistent with molecular formula C₂₀H₂₁FN₄O₂

### EXAMPLE 9

### N-(4-chloro-2-fluorophenyl)-6-methyl-4-(4-morpholinylcarbonyl)pyrrolo[1,2-a]pyrazin-1-amine hydrochloride

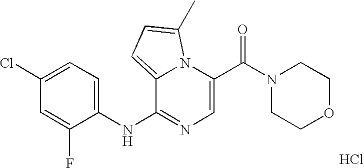

Prepared in a manner similar to Example 4 from 1-chloro-6-methyl-4-(4-morpholinylcarbonyl)pyrrolo[1,2-a]pyrazine (100 mg) (Intermediate 7) and 4-chloro-2-fluoroaniline (80 ul) to afford the title compound as a white solid (116 mg).

LC/MS [MH⁺] 389 consistent with molecular formula C₁₉H₁₈³⁵ClFN₄O₂

### EXAMPLE 10

### N-(5-Chloro-2-fluorophenyl)-6-methyl-4-(4-morpholinylcarbonyl)pyrrolo[1,2-a]pyrazin-1-amine hydrochloride

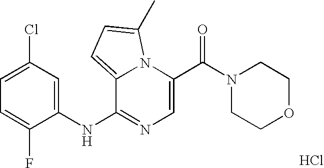

Prepared in a manner similar to Example 4 from 1-chloro-6-methyl-4-(4-morpholinylcarbonyl)pyrrolo[1,2-a]pyrazine (109 mg) (Intermediate 7) and 5-chloro-2-fluoroaniline (80 ul) to afford the title compound as a white solid (116 mg).

LC/MS [MH⁺] 389 consistent with molecular formula C₁₉H₁₈³⁵ClFN₄O₂

### EXAMPLE 11

### N-(3-chloro-4-fluorophenyl)-6-methyl-4-(4-morpholinylcarbonyl)pyrrolo[1,2-a]pyrazin-1-amine hydrochloride

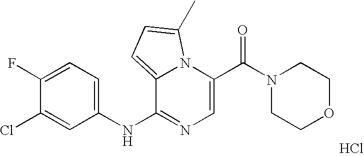

To a suspension of 1-chloro-6-methyl-4-(4-morpholinylcarbonyl)pyrrolo[1,2-a]pyrazine (50 mg) in 1,4-dioxane (0.75 ml) was added 3-chloro-4-fluoroaniline (52 mg) and methanesulfonic acid (23 ul) and the reaction mixture heated under microwave conditions at 180° C. for fifteen minutes. The solid mass was dissolved in methanol, transferred to a round bottomed flask and evaporated. The residue was purified by MDAP and the solid obtained suspended in methanol and treated with a few drops of 1M hydrochloric acid in diethyl ether to give a solution. The solution was evaporated and triturated with diethyl ether to give an off-white solid. The mixture was evaporated and dried to afford the title compound (49 mg).

LC/MS [MH⁺] 389 consistent with molecular formula C₁₉H₁₈³⁵ClFN₄O₂

### EXAMPLE 12

### N-(3,4-Difluorophenyl)-6-methyl-4-(4-morpholinylcarbonyl)pyrrolo[1,2-a]pyrazin-1-amine hydrochloride

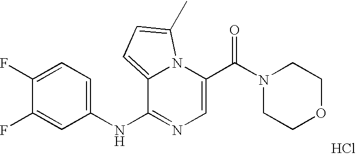

Prepared in a manner similar to Example 11 from 1-chloro-6-methyl-4-(4-morpholinylcarbonyl)pyrrolo[1,2-a]pyrazine (50 mg) (Intermediate 7) and 3,4-difluoroaniline (35 ul) to afford the title compound as a white solid (53 mg).

LC/MS [MH⁺] 373 consistent with molecular formula C₁₉H₁₈F₂N₄O₂

### EXAMPLE 13

### 6-Methyl-4-(4-morpholinylcarbonyl)-N-[3-(trifluoromethyl)phenyl]pyrrolo[1,2-a]pyrazin-1-amine hydrochloride

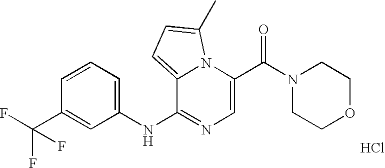

Prepared in a manner similar to Example 11 from 1-chloro-6-methyl-4-(4-morpholinylcarbonyl)pyrrolo[1,2-a]pyrazine (50 mg) (Intermediate 7) and 3-(trifluoromethyl)-aniline (45 ul) to afford the title compound as a white solid (48 mg).

LC/MS [MH⁺] 405 consistent with molecular formula C₂₀H₁₉F₃N₄O₂

### EXAMPLE 14

### N-(5-Bromo-2-fluorophenyl)-6-methyl-4-(4-morpholinylcarbonyl)pyrrolo[1,2-a]pyrazin-1-amine hydrochloride

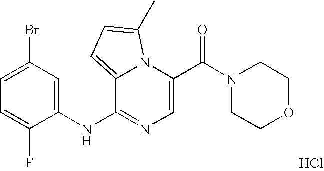

Prepared in a manner similar to Example 11 from 1-chloro-6-methyl-4-(4-morpholinylcarbonyl)pyrrolo[1,2-a]pyrazine (50 mg) (Intermediate 7) and 5-bromo-2-fluoroaniline (68 mg) to afford the title compound as a white solid (42 mg).

LC/MS [MH⁺] 435 consistent with molecular formula C₁₉H₁₈⁸¹BrFN₄O₂

### EXAMPLE 15

### N-(4-bromo-3-fluorophenyl)-6-methyl-4-(4-morpholinylcarbonyl)pyrrolo[1,2-a]pyrazin-1-amine hydrochloride

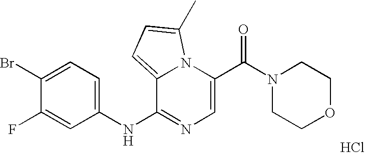

Prepared in a manner similar to Example 11 from 1-chloro-6-methyl-4-(4-morpholinylcarbonyl)pyrrolo[1,2-a]pyrazine (50 mg) (Intermediate 7) and 4-bromo-3-fluoroaniline (68 mg) except that after MDAP and prior to salt formation, it was triturated with diethyl ether to afford the title compound as a white solid (31 mg).

LC/MS [MH⁺] 435 consistent with molecular formula C₁₉H₁₈⁸¹BrFN₄O₂

### EXAMPLE 16

### N-(2,3-Dihydro-1-benzofuran-7-yl)-6-methyl-4-(4-morpholinylcarbonyl)pyrrolo[1,2-a]pyrazin-1-amine hydrochloride

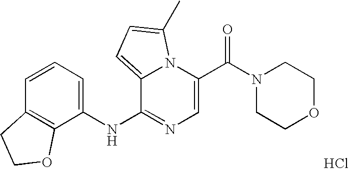

Prepared in a manner similar to Example 11 from 1-chloro-6-methyl-4-(4-morpholinylcarbonyl)pyrrolo[1,2-a]pyrazine (50 mg) (Intermediate 7) and 2,3-dihydro-1-benzofuran-7-amine (48 mg) (may be prepared according to Joiner, Graham Francis; Gaster, Laramie Mary. WO 95/11243) to afford the title compound as a white solid (63 mg).

LC/MS [MH⁺] 379 consistent with molecular formula C₂₁H₂₂N₄O₃

### EXAMPLE 17

### N-(5-Chloro-2,3-dihydro-1-benzofuran-7-yl)-6-methyl-4-(4-morpholinylcarbonyl)pyrrolo[1,2-a]pyrazin-1-amine hydrochloride

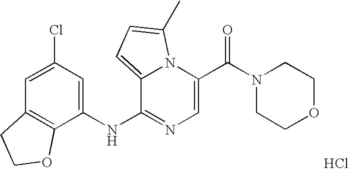

Prepared in a manner similar to Example 11 from 1-chloro-6-methyl-4-(4-morpholinylcarbonyl)pyrrolo[1,2-a]pyrazine (50 mg) (Intermediate 7) and 5-chloro-2,3-dihydro-1-benzofuran-7-amine (61 mg) (Intermediate 11) to afford the title compound as a white solid (52 mg).

LC/MS [MH⁺] 413 consistent with molecular formula C₂₁H₂₁³⁵ClN₄O₃

### EXAMPLE 18

### N-(5-Bromo-2,3-dihydro-1-benzofuran-7-yl)-6-methyl-4-(4-morpholinylcarbonyl)pyrrolo[1,2-a]pyrazin-1-amine hydrochloride

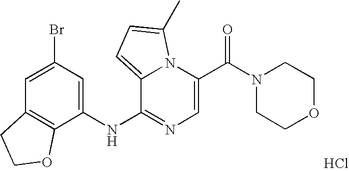

Prepared in a manner similar to Example 11 from 1-chloro-6-methyl-4-(4-morpholinylcarbonyl)pyrrolo[1,2-a]pyrazine (50 mg) (Intermediate 7) and 5-bromo-2,3-dihydro-1-benzofuran-7-amine (77 mg) (may be prepared according to Bromidge, Steven Mark; Moss, Stephen Frederik. WO 99/02502) to afford the title compound as a white solid (46 mg).

LC/MS [MH⁺] 459 consistent with molecular formula C₂₁H₂₁⁸¹BrN₄O₃

### EXAMPLE 19

### 1-[(3-Chlorophenyl)amino]-6-methyl-N-[2-(methyloxy)ethyl]pyrrolo[1,2-a]pyrazine-4-carboxamide hydrochloride

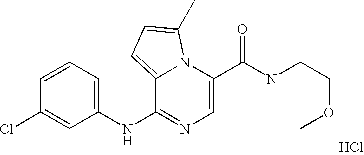

To 1-chloro-6-methyl-N-[2-(methyloxy)ethyl]pyrrolo[1,2-a]pyrazine-4-carboxamide (Intermediate 9 which contained 1-(1H-1,2,3-benzotriazol-1-yloxy)-6-methyl-N-[2-(methyloxy)ethyl]pyrrolo[1,2-a]pyrazine-4-carboxamide (15% by LCMS)) (53 mg) in 1,4-dioxane (1 ml) was added 3-chloroaniline (43 ul) and methanesulfonic acid (26 ul) and the reaction mixture heated under microwave conditions at 180° C. for fifteen minutes. The solid mass was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown oil. The oil was dissolved in dichloromethane, applied to a Biotage Si 25+S column and eluted with 10% ethyl acetate/hexane followed by 20% and 40% ethyl acetate/hexane to afford an off-white foam. The foam was dissolved in diethyl ether and treated with 1M hydrochloric acid in diethyl ether to afford a precipitate. The mixture was evaporated and dried to afford the title compound (43 mg).

LC/MS [MH⁺] 359 consistent with molecular formula C₁₈H₁₉³⁵ClN₄O₂

### EXAMPLE 20

### 1-[(3-Bromophenyl)amino]-6-methyl-N-[2-(methyloxy)ethyl]pyrrolo[1,2-a]pyrazine-4-carboxamide hydrochloride

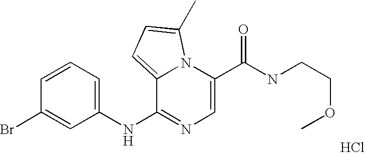

To 1-chloro-6-methyl-N-[2-(methyloxy)ethyl]pyrrolo[1,2-a]pyrazine-4-carboxamide (Intermediate 9 which contained 1-(1H-1,2,3-benzotriazol-1-yloxy)-6-methyl-N-[2-(methyloxy)ethyl]pyrrolo[1,2-a]pyrazine-4-carboxamide (15% by LCMS)) (53 mg) in 1,4-dioxane (1 ml) was added 3-bromoaniline (45 ul) and methanesulfonic acid (26 ul) and the reaction mixture heated under microwave conditions at 180° C. for fifteen minutes. The solid mass was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown oil. The oil was dissolved in dichloromethane, applied to a Biotage Si 25+S column and eluted with 10% ethyl acetate/hexane followed by 20% and 40% ethyl acetate/hexane to afford a white foam. The foam was dissolved in ethyl acetate and treated with 1M hydrochloric acid in diethyl ether to afford a precipitate. The mixture was evaporated and dried to afford the title compound (40 mg).

LC/MS [MH⁺] 405 consistent with molecular formula C₁₈H₁₉⁸¹BrN₄O₂

### EXAMPLE 21

### 6-Methyl-N-[2-(methyloxy)ethyl]-1-{[2-methyl-3-(trifluoromethyl)phenyl]amino}pyrrolo[1,2-a]pyrazine-4-carboxamide hydrochloride

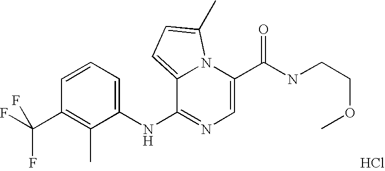

To 1-chloro-6-methyl-N-[2-(methyloxy)ethyl]pyrrolo[1,2-a]pyrazine-4-carboxamide (Intermediate 9 which contained 1-(1H-1,2,3-benzotriazol-1-yloxy)-6-methyl-N-[2-(methyloxy)ethyl]pyrrolo[1,2-a]pyrazine-4-carboxamide (15% by LCMS)) (53 mg) in 1,4-dioxane (1 ml) was added 3-amino-2-methylbenzotrifluoride (72 mg) and methanesulfonic acid (26 ul) and the reaction mixture heated under microwave conditions at 180° C. for fifteen minutes. The solid mass was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown oil. The oil was dissolved in dichloromethane, applied to a Biotage Si 25+S column and eluted with 10% ethyl acetate/hexane followed by 20%, 40% and 50% ethyl acetate/hexane to afford a pale yellow solid. The solid was dissolved in ethyl acetate and treated with 1M hydrochloric acid in diethyl ether to afford a precipitate. The mixture was evaporated and dried to afford the title compound (24 mg).

LC/MS [MH⁺] 407 consistent with molecular formula C₂₀H₂₁F₃N₄O₂

### EXAMPLE 22

### 1-[(3-Chlorophenyl)amino]-6-methyl-N-(tetrahydro-2H-pyran-4-ylmethyl)pyrrolo[1,2-a]pyrazine-4-carboxamide hydrochloride

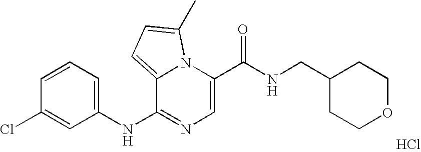

To 1-chloro-6-methyl-N-(tetrahydro-2H-pyran-4-ylmethyl)pyrrolo[1,2-a]pyrazine-4-carboxamide (Intermediate 8) (90 mg) in 1,4-dioxane (1.5 ml) was added 3-chloroaniline (62 ul) and methanesulfonic acid (38 ul) and the reaction mixture heated under microwave conditions at 180° C. for fifteen minutes. The solid mass was dissolved in methanol, transferred to a round bottomed flask and evaporated. The residue was purified by MDAP, triturated with diethyl ether and filtered. The solid obtained was suspended in methanol and treated with a few drops of 1M hydrochloric acid in diethyl ether to give a solution. The solution was evaporated and triturated with diethyl ether to give a white solid. The solid was filtered off and dried to afford the title compound (81 mg).

LC/MS [MH⁺] 399 consistent with molecular formula C₂₁H₂₃³⁵ClN₄O₂

### EXAMPLE 23

### 1-[(3-Bromophenyl)amino]-6-methyl-N-(tetrahydro-2H-pyran-4-ylmethyl)pyrrolo[1,2-a]pyrazine-4-carboxamide hydrochloride

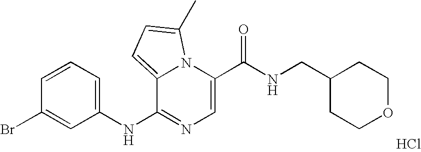

To Intermediate 8(a) (60 mg) in 1,4-dioxane (1 ml) was added 3-bromoaniline (43 ul) and methanesulfonic acid (25 ul) and the reaction mixture heated under microwave conditions at 180° C. for fifteen minutes. The reaction mixture was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown solid. The solid was purified by MDAP and the white solid obtained was suspended in methanol and treated with 1M hydrochloric acid in diethyl ether to give a solution. The solution was evaporated and co-evaporated from diethyl ether to give a white solid. The solid was transferred to a vial and dried to afford the title compound (45 mg).

LC/MS [MH⁺] 445 consistent with molecular formula C₂₁H₂₃⁸¹BrN₄O₂

### EXAMPLE 24

### 6-Methyl-N-(tetrahydro-2H-pyran-4-ylmethyl)-1-({3-[(trifluoromethyl)oxy]phenyl}amino)pyrrolo[1,2-a]pyrazine-4-carboxamide hydrochloride

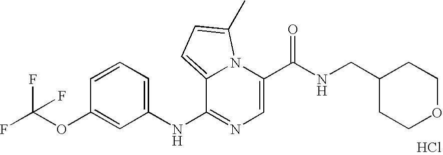

To Intermediate 8(a) (60 mg) in 1,4-dioxane (1 ml) was added 3-(trifluoromethoxy)aniline (52 ul) and methanesulfonic acid (25 ul) and the reaction mixture heated under microwave conditions at 180° C. for fifteen minutes. The reaction mixture was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown oil. The solid was purified by MDAP, triturated with diethyl ether and the off-white solid obtained was dissolved in methanol and treated with 1M hydrochloric acid in diethyl ether. The solution was evaporated and co-evaporated from diethyl ether to give a white solid. The solid was transferred to a vial and dried to afford the title compound (35 mg).

LC/MS [MH⁺] 449 consistent with molecular formula C₂₂H₂₃F₃N₄O₃

### EXAMPLE 25

### 6-Methyl-1-{[2-methyl-3-(trifluoromethyl)phenyl]amino}-N-(tetrahydro-2H-pyran-4-ylmethyl)pyrrolo[1,2-a]pyrazine-4-carboxamide hydrochloride

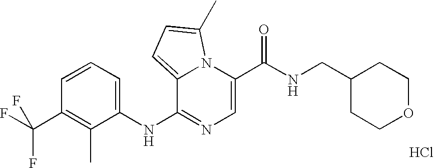

To Intermediate 8(a) (60 mg) in 1,4-dioxane (1 ml) was added 2-methyl-3-(trifluoromethyl)aniline (68 mg) and methanesulfonic acid (25 ul) and the reaction mixture heated under microwave conditions at 180° C. for fifteen minutes. The reaction mixture was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown oil. The oil was purified by MDAP and the white solid obtained was dissolved in methanol and treated with 1M hydrochloric acid in diethyl ether. The solution was evaporated and co-evaporated from diethyl ether to give a white solid. The solid was transferred to a vial and dried to afford the title compound (42 mg).

LC/MS [MH⁺] 447 consistent with molecular formula C₂₃H₂₅F₃N₄O₂

### EXAMPLE 26

### 1-[(4-bromo-3-fluorophenyl)amino]-6-methyl-N-(tetrahydro-2H-pyran-4-ylmethyl)pyrrolo[1,2-a]pyrazine-4-carboxamide hydrochloride

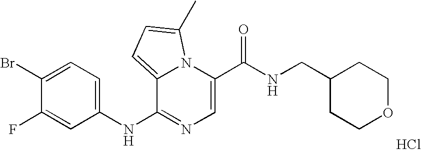

To Intermediate 8(a) (60 mg) in 1,4-dioxane (1 ml) was added 4-bromo-3-fluoroaniline (74 mg) and methanesulfonic acid (25 ul) and the reaction mixture heated under microwave conditions at 180° C. for fifteen minutes. The reaction mixture was dissolved in methanol, transferred to a round bottomed flask and evaporated. The residue was partitioned between ethyl acetate, water and saturated aqueous sodium hydrogen carbonate and washed. The organic layer was dried (MgSO₄) and evaporated to a brown solid. The solid was purified by MDAP, triturated with diethyl ether and the off-white solid obtained was suspended in methanol and treated with 1M hydrochloric acid in diethyl ether. The mixture was evaporated, triturated with diethyl ether and filtered off to give a pink solid. The solid was transferred to a vial and dried to afford the title compound (37 mg).

LC/MS [MH⁺] 463 consistent with molecular formula C₂₁H₂₂⁸¹BrFN₄O₂

### EXAMPLE 27

### 1-{[2-Fluoro-3-(trifluoromethyl)phenyl]amino}-6-methyl-N-(tetrahydro-2H-pyran-4-ylmethyl)pyrrolo[1,2-a]pyrazine-4-carboxamide hydrochloride

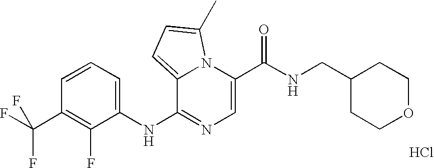

To Intermediate 8(b) (150 mg) in 1,4-dioxane (1.5 ml) was added 2-fluoro-3-(trifluoromethyl)aniline (103 ul) and methanesulfonic acid (52 ul) and the reaction mixture heated under microwave conditions at 180° C. for fifteen minutes. The reaction mixture was dissolved in methanol, transferred to a round bottomed flask and evaporated. The residue was dissolved in ethyl acetate/water, transferred to a separating funnel and saturated aqueous sodium hydrogen carbonate added and washed. The organic layer was dried (MgSO₄) and evaporated to a brown oil. The oil was purified by MDAP and the pale yellow solid obtained was suspended in methanol and treated with 1M hydrochloric acid in diethyl ether to give a solution. The solution was evaporated and co-evaporated from diethyl ether to afford the title compound (42 mg) as a pale yellow solid.

LC/MS [MH⁺] 451 consistent with molecular formula C₂₂H₂₂F₄N₄O₂

### EXAMPLE 28

### 1-{[5-Fluoro-2-(methyloxy)phenyl]amino}-6-methyl-N-(tetrahydro-2H-pyran-4-ylmethyl)pyrrolo[1,2-a]pyrazine-4-carboxamide hydrochloride

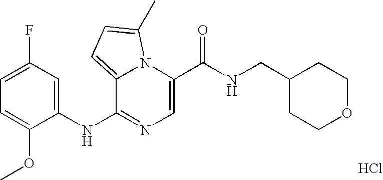

Prepared in a manner similar to Example 27 from Intermediate 8(b) (150 mg) and 5-fluoro-2-methoxyaniline (113 mg) to afford the title compound as a pale brown solid (43 mg).

LC/MS [MH⁺] 413 consistent with molecular formula C₂₂H₂₅FN₄O₃

### EXAMPLE 29

### 1-[(3-Fluoro-4-methylphenyl)amino]-6-methyl-N-(tetrahydro-2H-pyran-4-ylmethyl)pyrrolo[1,2-a]pyrazine-4-carboxamide hydrochloride

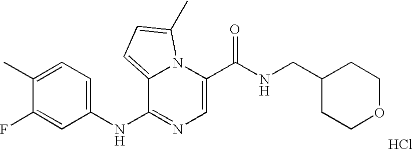

Prepared in a manner similar to Example 27 from Intermediate 8(b) (150 mg) and 3-fluoro-4-methylaniline (100 mg) to afford the title compound as a pale brown solid (53 mg).

LC/MS [MH⁺] 397 consistent with molecular formula C₂₂H₂₅FN₄O₂

### EXAMPLE 30

### 1-[(4-Chloro-2-fluorophenyl)amino]-6-methyl-N-(tetrahydro-2H-pyran-4-ylmethyl)pyrrolo[1,2-a]pyrazine-4-carboxamide hydrochloride

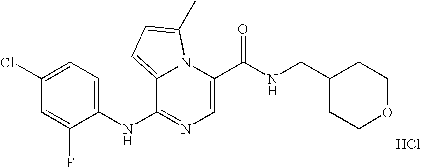

Prepared in a manner similar to Example 27 from Intermediate 8(b) (150 mg) and 4-chloro-2-fluoroaniline (90 ul) to afford the title compound as a white solid (16 mg).

LC/MS [MH⁺] 417 consistent with molecular formula C₂₁H₂₂³⁵ClFN₄O₂

### EXAMPLE 31

### 1-[(5-Chloro-2-fluorophenyl)amino]-6-methyl-N-(tetrahydro-2H-pyran-4-ylmethyl)pyrrolo[1,2-a]pyrazine-4-carboxamide hydrochloride

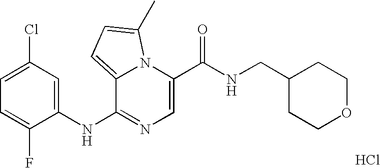

Prepared in a manner similar to Example 27 from Intermediate 8(b) (150 mg) and 5-chloro-2-fluoroaniline (116 mg) to afford the title compound as a yellow solid (25 mg).

LC/MS [MH⁺] 417 consistent with molecular formula C₂₁H₂₂³⁵ClFN₄O₂

### EXAMPLE 32

### 1-[(3-Chloro-4-fluorophenyl)amino]-6-methyl-N-(tetrahydro-2H-pyran-4-ylmethyl)pyrrolo[1,2-a]pyrazine-4-carboxamide hydrochloride

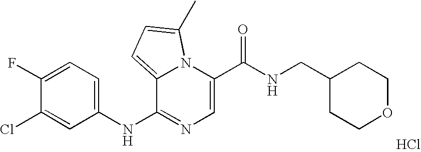

Prepared in a manner similar to Example 27 from Intermediate 8(b) (150 mg) and 3-chloro-4-fluoroaniline (116 mg) to afford the title compound as a yellow solid (25 mg).

LC/MS [MH⁺] 417 consistent with molecular formula C₂₁H₂₂³⁵ClFN₄O₂

### EXAMPLE 33

### 1-[(3,4-Difluorophenyl)amino]-6-methyl-N-(tetrahydro-2H-pyran-4-ylmethyl)pyrrolo[1,2-a]pyrazine-4-carboxamide hydrochloride

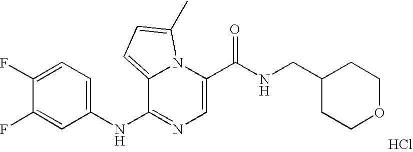

Prepared in a manner similar to Example 27 from Intermediate 8(b) (150 mg) and 3,4-difluoroaniline (80 ul) to afford the title compound as a pale brown solid (56 mg).

LC/MS [MH⁺] 401 consistent with molecular formula C₂₁H₂₂F₂N₄O₂

### EXAMPLE 34

### 6-Methyl-N-(tetrahydro-2H-pyran-4-ylmethyl)-1-{[3-(trifluoromethyl)phenyl]amino}pyrrolo[1,2-a]pyrazine-4-carboxamide hydrochloride

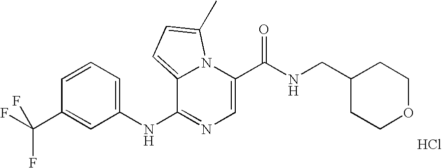

Prepared in a manner similar to Example 27 from Intermediate 8(b) (150 mg) and 3-(trifluoromethylaniline (100 ul) to afford the title compound as an off-white solid (50 mg).

LC/MS [MH⁺] 433 consistent with molecular formula C₂₂H₂₃F₃N₄O₂

Formulations for pharmaceutical use incorporating compounds of the present invention can be prepared in various forms and with numerous excipients. Examples of such formulations are given below.

### EXAMPLE 35

### Inhalant Formulation

A compound of formula (I) or a pharmaceutically acceptable derivative thereof, (1 mg to 100 mg) is aerosolized from a metered dose inhaler to deliver the desired amount of drug per use.

### EXAMPLE 36

### Tablet Formulation

| Tablets/Ingredients | | Per Tablet |
|---|---|---|
| 1. | Active ingredient | 40 mg |
| | (Compound of formula (I) or | |
| | pharmaceutically | |
| | acceptable derivative) | |
| 2. | Corn Starch | 20 mg |
| 3. | Alginic acid | 20 mg |
| 4. | Sodium Alginate | 20 mg |
| 5. | Mg stearate | 1.3 mg |

### Procedure for Tablet Formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 37

### Parenteral Formulation

A pharmaceutical composition for parenteral administration is prepared by dissolving an appropriate amount of a compound of formula (I) in polyethylene glycol with heating. This solution is then diluted with water for injections Ph Eur. (to 100 ml). The solution is then rendered sterile by filtration through a 0.22 micron membrane filter and sealed in sterile containers.

What is claimed is:

1. A compound of formula (I): 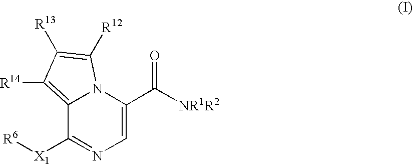
wherein:
X₁ is NR⁴, O, S, SO or SO₂;
R¹ is selected from hydrogen, C₁₋₆ alkyl, C₃₋₆ cycloalkyl and halosubstitutedC₁₋₆ alkyl;
R² is hydrogen or (CH₂)ₘR³ where m is 0 or 1;
or R¹ and R² together with N to which they are attached form an optionally substituted 4- to 8-membered non-aromatic heterocyclyl ring;
R³ is a 4- to 8-membered non-aromatic heterocyclyl group, a C₃₋₈ cycloalkyl group, a straight or branched C₁₋₁₀ alkyl, a C₂₋₁₀alkenyl, a C₃₋₈cycloalkenyl, a C₂₋₁₀alkynyl, a C₃₋₈cycloalkynyl or phenyl group, any of which can be unsubstituted or substituted, or R⁵;
R⁴ is selected from hydrogen, C₁₋₆ alkyl, C₃₋₆ cycloalkyl and halosubstitutedC₁₋₆ alkyl;
R⁵ is 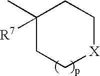
wherein p is 0, 1 or 2, and X is CH₂, O, S, or SO₂;
R⁶ is unsubstituted or substituted phenyl, unsubstituted or substituted C₃₋₆cycloalkyl or an unsubstituted or substituted 4- to 8-membered non-aromatic heterocyclyl ring;
R⁷ is OH;
R¹² is hydrogen or C₁₋₆alkyl;
R¹³ is hydrogen or C₁₋₆alkyl;
R¹⁴ is hydrogen or C₁₋₆alkyl;
or a pharmaceutically acceptable derivative thereof.

2. A compound as claimed in claim 1 wherein R¹ is hydrogen.

3. A compound as claimed in claim 1 wherein R² is (CH₂)ₘR³ where m is 0 or 1.

4. A compound as claimed in claim 1 wherein R³ is an unsubstituted or substituted C₁₋₆ alkyl group or tetrahydropyranyl.

5. A compound as claimed in claim 1 wherein R¹ and R² together with the nitrogen to which they are attached form a morpholinyl, pyrrolidinyl or piperidinyl ring.

6. A compound as claimed in claim 1
wherein R⁶ is an unsubstituted or substituted phenyl group.

7. A compound as claimed in claim 1 wherein X₁ is NR⁴.

8. A compound as claimed in claim 1
wherein R⁴ is C₁₋₆ alkyl or hydrogen.

9. A compound as claimed in claim 8 wherein R⁴ is methyl.

10. A compound as claimed in claim 1
wherein R¹² is methyl.

11. A compound as claimed in claim 1
wherein R¹³ is hydrogen.

12. A compound as claimed in claim 1
wherein R¹⁴ is hydrogen.

13. A compound of formula (Ia): 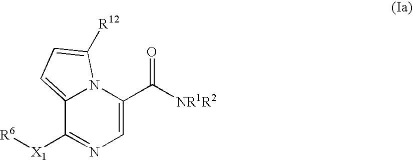
wherein
X₁ is NR⁴;
R¹ is hydrogen;
R² is (CH₂)ₘR³ where m is 0 or 1;
or R¹ and R² together with N to which they are attached form a morpholinyl, pyrrolidinyl, or piperidinyl ring of which may be unsubstituted or substituted;
R³ is an unsubstituted or substituted straight or branched C₁₋₆ alkyl;
R⁴ is hydrogen or methyl,
R⁶ is unsubstituted or substituted phenyl;
R¹² is hydrogen or methyl;
or a pharmaceutically acceptable derivative thereof.

14. (canceled)

15. (canceled)

16. A pharmaceutical composition comprising a compound as claimed in claim 1 or a pharmaceutically acceptable derivative thereof.

17. A pharmaceutical composition as claimed in claim 16 further comprising a pharmaceutical carrier or diluent thereof.

18. A pharmaceutical composition as claimed in claim 17 further comprising a second therapeutic agent.

19. (canceled)

20. (canceled)

21. A method of treating mammal suffering from a condition which is mediated by the activity of cannabinoid 2 receptor which comprises administering to said subject a therapeutically effective amount of a compound of formula (I) as claimed in claim 1 or a pharmaceutically acceptable derivative thereof.

22. A method for treating a condition selected from an immune disorder, an inflammatory disorder, pain, rheumatoid arthritis, multiple sclerosis, osteoarthritis or osteoporosis said method comprising administering to the mammal a therapeutically effective amount of a compound according to claim 1.

23. The method as claimed in claim 22, wherein the pain is selected from inflammatory pain, visceral pain, cancer pain, neuropathic pain, lower back pain, muscular skeletal, post operative pain, acute pain and migraine.

24. The method of treating of claim 21, wherein the mammal is a human.
